(19) United States
(12) Patent Application Publication
(10) Pub. No.: US 2023/0399286 A1
(43) Pub. Date: Dec. 14, 2023

(54) IMPROVED PROCESS FOR PREPARING METHYL METHACRYLATE AND/OR METHACRYLIC ACID BY REDUCED BACK MIXING DURING CONVERSION

(71) Applicant: Röhm GmbH, Darmstadt (DE)

(72) Inventors: Ufuk KOCKESEN, Niederkassel (DE); Patrick Wings, Koeln (DE); Steffen Krill, Muehtal (DE); Florian Klasovsky, Haltern am See (DE)

(73) Assignee: Röhm GmbH, Darmstadt (DE)

(21) Appl. No.: 18/249,917

(22) PCT Filed: Oct. 7, 2021
(86) PCT No.: PCT/EP2021/077640

§ 371 Date: Apr. 20, 2023

(30) Foreign Application Priority Data
Oct. 23, 2020 (EP) 20203722.2

(51) Int. Cl. C07C 69/54; C07C 67/20; C07C 51/06; C07C 57/04; C07C 231/06; C07C 231/12; C07C 303/24

(52) U.S. Cl. CPC C07C 69/54; C07C 67/20; C07C 51/06; C07C 57/04; C07C 231/06; C07C 231/12; C07C 303/24

(57) ABSTRACT

A process for preparing methyl methacrylate (MMA) and/or methacrylic acid (MAS) having improved yield, involves amidation, conversion, and hydrolysis/esterification. Especially high yields are obtained during the amidation and in the subsequent so-called conversion.

The present invention relates to a process for preparing methyl methacrylate (MMA) and/or methacrylic acid (MA) having improved yield, comprising the process steps of amidation, conversion and hydrolysis/esterification, wherein high yields are achieved especially in the step of amidation (also referred to as hydrolysis of acetone cyanohydrin) and in the subsequent process step, called conversion. The conversion comprises the reaction of the reaction mixtures in sulfuric acid that are obtained in the amidation, containing a high proportion of α-sulfoxyisobutyramide (SIBA), to give a reaction mixture comprising methacrylamide (MAA) in sulfuric acid. The inventive construction of the reactors used in the conversion step, especially thermal conversion apparatuses, in conjunction with an optimized concentration of the sulfuric acid used enables a distinct gain in yield of MAA compared to the prior art. In addition, optimization can be achieved with the aid of cooling of the reaction mixture which is effected between conversion and esterification. The optimized MAA yields are additionally subject to much smaller variations and result in an elevated overall yield to give MMA or MA. The yields can additionally exceed the yields achievable by other processes, even in the case of operation at partial load.

The invention encompasses specific flow-optimized apparatus constructions in the conversion step in combination with the optimal adjustment of stoichiometric process parameters, especially the monitoring and optimization of the sulfuric acid concentration.

### PRIOR ART

There are multiple known commercial processes for preparing alkyl methacrylates, especially MMA, on an industrial scale. A commercial process in global use is based on acetone as raw material, and is usually referred to as C3 process or ACH-sulfo process. Acetone is reacted here with hydrogen cyanide (HCN) to give the central acetone cyanohydrin (ACH) intermediate. This intermediate is isolated and used for the subsequent process steps for preparation of MA and MMA.

A typical process for preparing MMA proceeding from ACH is described, for example, in U.S. Pat. No. 4,529,816. A multitude of further documents describes how the various aspects of the reaction steps can be improved and optimized, the ultimate aim being to increase the overall yield of the desired MA and MMA monomers.

In the ACH-sulfo process, ACH is hydrolysed with the aid of sulfuric acid to form α-hydroxyisobutyramide (HIBAm) and its sulfate ester (SIBA). This step is referred to in the relevant literature as amidation or else hydrolysis, since the nitrile group of the ACH is formally converted to an amide function.

HIBAm, SIBA, and MAA already formed in the amidation are subsequently converted thermally to MAA and relatively small amounts of MA in the reaction mixture in sulfuric acid. This step is referred to in the relevant literature as conversion, wherein the sulfoxy group of the SIBA is converted in a formal sense to an olefinic double bond function by a β-elimination, wherein MAA is formed from SIBA, and free sulfuric acid is released again in a formal sense.

The MAA solution in sulfuric acid obtained after conversion can then be admixed with methanol and water in order to prepare MMA by an esterification reaction. Alternatively, the MAA solution in sulfuric acid obtained after the conversion can be reacted with water to give methacrylic acid (MA) (hydrolysis). Residual HIBAm is typically converted here partly to methyl hydroxyisobutyrate (MHIB), but hydrolysed partly also to hydroxyisobutyric acid (HIBAc).

In the conversion. SIBA is more easily converted to MAA than HIBAm is converted to MAA. In order to accelerate the thermal conversion of HIBAm to MAA, both heat and a longer dwell time would have to be provided. However, this process is again associated with degradation of yield-relevant intermediates such as MAA, wherein tar-like deposits can be formed, as can a multitude of compounds formed consecutively that contribute to a reduction in yield. The reaction conditions therefore have to be optimized such that, in particular, the desired main reaction of the conversion, namely the conversion of SIBA→MAA, and the likewise desired conversion of the HIBAm by-product to MAA are promoted. A decrease in the thermal conversion to desired products typically leads to a reduced overall yield for the process.

The process for preparing MA has essentially similar features to the preparation of MMA. In the preparation of MA and the preparation of MMA, amidation and conversion are often fundamentally similar or identical, with a maximum MAA yield from ACH being desirable in both cases. If the MAA-containing reaction mixture is reacted solely with water without alcohol, the reaction (hydrolysis of MAA) typically leads to crude MA mixtures. By contrast, the reaction (esterification) of the MAA-containing reaction mixture in the presence of water and alcohol (e.g. methanol) leads to alkyl methacrylates (e.g. MMA). It is known to the person skilled in the art in this connection that, for the selective preparation of MA, it is necessary to provide higher molar proportions of water than is the case for the preparation of MMA.

Both the preparation of MA and the production of MMA are conducted in plant complexes that often produce up to several hundreds of thousands of tonnes of product per year. In this respect, even slight improvements in the yield can lead to significant savings. A side effect which is not to be underestimated in respect of the ecological footprint of the processes additionally lies in the assessment of the waste materials in these processes. In general, considerable amounts of waste acid are generated, which often have to be regenerated with a high level of energy expenditure, in order to recover the sulfuric acid as starting material. Tar-like impurities in the waste acid generally have to be removed partly manually from time to time and disposed of at great cost and inconvenience, which especially makes high demands on technology and operational reliability.

The problem of controlling dwell time in tubular reactors at high temperatures that permit conversions of matter has been described in a multitude of patents and publications in the prior art. For example. EP 0 202 099 A discloses a process for treatment of heavy oil residues. Heavy oil residues are subjected here to thermal cracking, using a thermal reactor consisting of tubes and having a plug flow profile. The resulting cracking products are removed by means of stripping.

U.S. Pat. No. 5,393,918 describes an approach for improving the yield of an MMA preparation process. However, there is no improvement here in the conversion step or the MAA yield, but rather the transformation of a by-product of the esterification, namely methyl hydroxyisobutyrate (MHIB), to give additional amounts of MMA. U.S. Pat. No. 5,393,918 describes a process in which HIBAm and SIBA, in the esterification step that follows the amidation, are converted to the two configurational isomers of methyl methoxyisobutyrate (α-MMIB and β-MMIB). These three by-products are very substantially separated from MMA in the MMA workup and converted to MMA in a separate dehydration step. This complex process regime eliminates the need for a thermal conversion of HIBAm and SIBA to MAA, but entails a fractional distillation and a subsequent additional dehydration step. The process according to U.S. Pat. No. 5,393,918 is thus complex from a technical point of view, laborious, and requires additional apparatuses and reactors.

The thermal conversion of HIBAm and SIBA to MAA is typically conducted in tubular reactors that are partly referred to as cracking reactors in the prior art. This name illustrates the thermally demanding conditions and the desired β-elimination reaction. These reactors for performance of the conversion generally contain, as apparatus elements, at least one heat exchanger for provision of the energy required for the reaction, and delay apparatuses that provide the required dwell time for the MAA formation under the required conditions.

A typical thermal conversion apparatus and apparatus embodiment of a conversion reactor are described, for example, in EP 0 999 200. The conversion reactor described here comprises a metal tube with multiple passages and openings. In one embodiment, the metal tube may comprise a dividing wall that divides the tube in order to provide for passage with a pipe bend element (180° diversion of the reaction mixture). The configuration of the tubular reactor with deflections often serves to minimize the space requirement of the conversion reactor. The reactor in which the conversion of the SIBA-containing amidation mixture to MAA is conducted may, for example, be composed of tubular trace-heated elements and deflecting pipe elements that are optionally connected to one another with flanges. The conversion reactor described here additionally widens at the point where the amidation mixture enters the thermal conversion apparatus, and narrows at the point where the conversion mixture leaves the thermal conversion apparatus.

Various problems are apparent in the prior art, for example in EP 0 999 200. According to EP 0 999 200, yields after the amidation step of 96% to 97% and yields after the conversion step in the order of magnitude of 92% to 94% are achieved. What are especially noticeable are fluctuations in yield by up to 2%, such that it is not possible to maintain sufficiently stable process operation on an industrial scale with the measures described. The yields described in EP 0 999 200 are achieved by a specific conversion procedure with assurance of plug flow and a basic scrubbing method for the esterification mixture. In principle, the configuration in EP 0 999 200 entails a high level of apparatus complexity.

These construction features of a typical thermal conversion apparatus lead to backmixing of HIBAm, SIBA, MAA and MA that are preformed in the amidation mixture from ACH. One reason why the backmixing of these intermediates occurs is changes in speed at the pipe transitions. More particularly, disruption of plug flow leads to a reduced overall yield since there will be variation in the dwell time of the components in the conversion reactor. Plug flow, as is known to the person skilled in the art, is characterized by a parabolic speed profile in which the molecules at the inner wall of the tube have a lower speed than molecules in the core of the flow or in the middle of the tube. With increasing flow through the pipeline (and rising Reynolds number), the parabola of the speed distribution over the tube cross section flattens out. The prior art does not sufficiently describe the relationship between the flow regime (e.g. the influence of the Reynolds number) and the progression of the amidation reaction.

Inadequate and/or varying yields in the conversion reactor often have the following cause: In statistical terms, particular proportions of the components present in the reaction mixture have a dwell time in the conversion reactor that is much greater or much less than the dwell time needed for the full performance of the target reactions. While excessively short dwell times often result in incomplete conversion of SIBA or HIBAm to MAA, high dwell times often have the effect that the above-described thermal degradation products are formed. Both forms of inadequate control of dwell time in the conversion reactor thus typically contribute to lowering of the achievable yield of target product.

The advantageous use of a tubular reactor to provide a plug flow in the preparation of methacrylic esters is described in U.S. Pat. No. 4,748,268 A. What is described here is how a stream of matter comprising methacrylic acid, a C₁-C₄ alcohol, a catalyst and a liquid organic substance is fed into a plug flow apparatus. However, this description merely encompasses the esterification of methacrylic acid, and not the formation of methacrylic esters from HIBAm and SIBA comprising a conversion step.

Thus, there is still a need for a commercial process having improved yield for preparation of MMA or MAA, especially in the steps of amidation (hydrolysis) and conversion.

### Problem

The problem addressed by the present invention was that of sustainably increasing the yield of the ACH-sulfo process for preparing methacrylamide and its methyl methacrylate and methacrylic acid conversion products with a low level of expenditure and a simultaneously high level of operational reliability. It was a particular object of the present invention to minimize backmixing phenomena in the thermal conversion steps in the preparation of MMA, and to reduce the formation of by-products and hence of waste.

### Solution

The problem was solved in that, in the process according to the invention, the minimization of backmixing and the targeted control of dwell time in the conversion significantly improve the thermal conversion of HIBAm and SIBA to MAA, and therefore give an elevated overall process yield of the preparation process. More particularly, backmixing is reduced by the very substantial maintenance of what is called plug flow, plug flow meaning that the speed of the fluid, for example of the reaction mixture, in the tube is virtually constant over the entire tube cross section. It has been found that a novel combination of apparatuses for flow optimization and process parameters, such as, in particular, the concentration of the sulfuric acid used, can give an optimized yield in the conversion and hence also in the overall process.

It has additionally been found that, surprisingly, in the event of excessive thermal stress and excessively long dwell times, not inconsiderable amounts of MAA dimers and oligomers are formed, as are mixed dimers and oligomers formed from MAA and MA. For achievement of a high MMA yield, the minimization of the concentration of free MA in the conversion mixture is also desirable, since this likewise has a tendency to unwanted further reactions under the thermal conditions of the conversion.

By fluid-dynamic simulation of flow and mixing, it was possible to visualize the weak points in the conversion process step and especially the variances from desired parameters (for example in the dwell time) from the fixed ideal conditions. By the adjustments according to the invention in the construction of the conversion reactor, especially the thermal conversion apparatus, it was possible to remedy these weak points. More particularly, it is possible through specific combination of reduced backmixing and a uniformly distributed flow profile in the conversion step of the process to significantly improve the thermal conversion of SIBA and HIBAm to MAA, and hence to achieve the desired increase in yield of MMA or MA.

The invention and its characterizing features are described in detail hereinafter.

### DESCRIPTION OF THE INVENTION

The present invention relates to a process for preparing methyl methacrylate and/or methacrylic acid, comprising the process steps of:
a. the reacting of acetone cyanohydrin and sulfuric acid in one or more reactors I in a first reaction stage (amidation) at a temperature in the range from 70° C. to 130° C. to obtain a first reaction mixture comprising sulfoxyisobutyramide (SIBA) and methacrylamide (MAA);
b. the converting of the first reaction mixture, comprising heating to a temperature in the range from 130 to 200° C. in one or more reactors II in a second reaction stage (conversion) to obtain a second reaction mixture comprising predominantly methacrylamide and sulfuric acid; and
c. the reacting of the second reaction mixture with water and optionally methanol in one or more reactors III in a third reaction stage (esterification or hydrolysis) to obtain a third reaction mixture comprising methacrylic acid and/or methyl methacrylate;
wherein
   (i) the sulfuric acid used in the first reaction stage, which is fed in at one or more points in the reactors I, has a concentration in the range from 98.0% by weight to 100.5% by weight,
   (ii) the dwell time of the first reaction mixture in the second reaction stage is in the range from 2 to 15 min,
   (iii) the heating in the second reaction stage is performed in one or more reactors II, wherein at least one reactor II comprises at least one preheater segment comprising one or more linear pipeline elements that are heated by means of a heating medium spatially separate from the reaction mixture, wherein the reaction mixture is heated by 10 to 100° C.,
   (iv) the converting in the second reaction stage is performed in one or more reactors II, wherein at least one reactor II comprises at least one delay segment which is operated under virtually adiabatic conditions,
   (v) the preheater segment and/or the delay segment are implemented in a combination of linear pipeline elements and deflections, wherein the linear pipeline elements and the deflections are connected to one another via reducing flanges,
   (vi) the second reaction mixture obtained in step b, comprising predominantly methacrylamide and sulfuric acid, is optionally cooled down to a temperature below 120° C., for example in a cooler with a cooling medium having a temperature in the range from 60 to 100° C., and/or optionally intermediately buffered in an intermediate vessel, before the reaction mixture is guided into the third reaction stage.

The present invention additionally relates to an improved process for preparing methyl methacrylate and/or methacrylic acid, comprising (a) the reacting of acetone cyanohydrin (ACH) and sulfuric acid in a first reaction stage (amidation) to obtain a first reaction mixture comprising at least hydroxyisobutyramide (HIBAm), sulfoxyisobutyramide (SIBA), methacrylic acid (MA) and methacrylamide (MAA), and (b) the heating of the first reaction mixture in a second reaction stage (conversion), giving primarily methacrylamide (MAA), and (c) the subsequent esterification of methacrylamide (MAA) with methanol and water to form methyl methacrylate or (c) the subsequent hydrolysis of methacrylamide (MAA) with water to form methacrylic acid (MA) in a third reaction stage (esterification or hydrolysis), wherein the sulfuric acid used in the first reaction stage has a concentration in the range from 98.0% by weight to 100.5% by weight, and wherein at least the step of the conversion is performed in a thermal conversion apparatus that assures a plug flow profile with minimal backmixing and allows exact control and adjustment of the dwell time in the process steps mentioned.

More particularly, the present invention relates to an optimized process for preparing methyl methacrylate and/or methacrylic acid, comprising the specific adjustment and control of the dwell time in the steps of amidation and/or conversion, especially of conversion, wherein firstly the inadequate conversion of yield-relevant intermediates such as SIBA and HIBAm as a result of excessively low dwell time and secondly the yield-reducing breakdown of reactive intermediates as a result of excessively high dwell time in the process steps mentioned are suppressed, and the yield of the MAA and MA products, and hence ultimately also of MMA, is simultaneously improved.

In the context of the present invention, the expression “ppm” without further qualifiers means ppm by weight (e.g. mg/kg).

The expression “stream, phase or fraction comprising a reactant, product and/or by-product” is understood in the context of the invention to mean that the compound(s) mentioned is/are present in the respective stream; for example, the predominant proportion of the reactant, product and/or by-product is to be found in the corresponding stream. In principle, further constituents may be present as well as the compounds mentioned. The naming of the constituents often serves to illustrate the respective process step.

The expression “vapour” or “vapour stream” in the context of the invention refers to a gaseous process stream, for example a gaseous top stream from a distillation column.

In the context of the invention, the expression “adiabatic” means that negligible heat exchange, if any, takes place with the environment.

First Reaction Stage (Amidation)

The process according to the invention comprises, as step (a), the reacting of acetone cyanohydrin (ACH) and sulfuric acid in one or more reactors I in a first reaction stage (amidation) at a temperature in the range from 70 to 130° C., preferably 70 to 120° C., to obtain a first reaction mixture comprising sulfoxyisobutyramide and methacrylamide.

According to the invention, the sulfuric acid used in the first reaction stage has a concentration in the range from 98.0% by weight to 100.5% by weight, preferably 98.0% by weight to 100.0% by weight, preferably of 99.0% by weight to 99.9% by weight, preferably of 99.3% by weight to 99.9% by weight, especially preferably of 99.3% to 99.8% by weight. More particularly, the stated concentration of the sulfuric acid used is based on the total mass of the sulfuric acid feed stream to the first reaction stage (e.g. (2)). The use of a sulfuric acid having a zero content of free SO₃, especially a sulfuric acid with a water content of 0.2% to 0.7% by weight, has been found to be particularly advantageous.

The person skilled in the art is aware in principle of methods of determining the water content of streams of matter, for example of sulfuric acid feed streams. For example, the water content of streams of matter can be ascertained by mass balances, by measuring the density or speed of sound, by gas chromatography, by Karl Fischer titration, or by means of HPLC.

The ACH used can be prepared by means of known industrial processes (see, for example, Ullmanns Enzyklopädie der technischen Chemie [Ullmann's Encyclopedia of Industrial Chemistry], 4th edition, volume 7). Typically, hydrogen cyanide and acetone are converted to ACH in an exothermic reaction in the presence of a basic catalyst, for example an amine. Such a process stage is described, for example, in DE 10 2006 058 250 and DE 10 2006 059 511.

Typically, the amidation of ACH and sulfuric acid forms, as main products, α-hydroxyisobutyramide (HIBAm) or its hydrogensulfate (HIBAm·H₂SO₄), sulfuric esters of HIBAm (α-sulfoxyisobutyramide, SIBA) or its hydrogensulfate (SIBA·H₂SO₄) and methacrylamide hydrogensulfate (MAA·H₂SO₄), as a solution in excess sulfuric acid. It is known to the person skilled in the art that the proportions of the components mentioned in the reaction mixture are variable and depend on the reaction conditions.

The first reaction stage is preferably conducted with an excess of sulfuric acid. The sulfuric acid preferably serves as solvent. At the same time, the sulfuric acid serves as reactant (for the forming of the SIBA intermediate) and as catalyst for the conversion. The sulfuric acid excess can especially serve to keep the viscosity of the reaction mixture low, which can assure faster removal of heat of reaction and a lower temperature of the reaction mixture. This can especially bring distinct yield benefits. Even though viscosity and dissolution capacity are improved with more sulfuric acid, which ultimately also entails a general increase in selectivity, the upper limit on the amount of sulfuric acid used is ultimately for economic reasons since the resulting volume of waste acid has to be recycled or processed further.

In a preferred embodiment, the reaction mixture composed of acetone cyanohydrin (ACH) and sulfuric acid includes, in the first reaction mixture, a total amount of water in the range from 0.1 mol % to 20 mol %, especially 0.4 mol % to 10 mol %, based on the overall ACH supplied to the first reaction stage. Preference is given to using acetone cyanohydrin (ACH) in the first reaction stage, wherein the ACH or the ACH streams supplied (e.g. (1a) and/or (1b)) have an acetone content of not more than 9000 ppm, preferably of not more than 1000 ppm, based on the total amount of ACH which is supplied to the first reaction stage. Preferably, the ACH used has, or the ACH streams supplied (e.g. (1a) and/or (1b)) have, an ACH content of not less than 98% by weight, more preferably not less than 98.5% by weight, especially preferably not less than 99% by weight, based on the ACH streams supplied. Typically, the ACH stream supplied (e.g. (1a) and/or (1b)) contains 98.0% to 99.8% by weight, preferably 98.3% to 99.3% by weight, of acetone cyanohydrin, 0.1% to 1.5% by weight, preferably 0.2% to 1% by weight, of acetone, and 0.1% to 1.5% by weight, preferably 0.3% to 1% by weight, of water, based on the ACH stream.

Preferably, in the first reaction stage, acetone cyanohydrin (ACH) is used, wherein the ACH has, or the ACH streams supplied (e.g. (1a) and/or (1b)) have, a water content of 0.1 mol % to 10 mol %, especially 0.4 mol % to 5 mol %, based on the ACH present in the ACH streams supplied.

For performance of the amidation (hydrolysis) in the first reaction stage, it is possible in principle to use any reactor known to the person skilled in the art for the performance of hydrolysis reactions, for example stirred tank reactors and loop reactors or combinations of said reactors. Alternatively, it is possible to use a plurality of reactors, optionally connected in parallel, but preferably in series. In one possible embodiment, 1 to 5 reactors are connected in series; preference is given to a sequential arrangement of 2-3 reactors.

Preference is given to using sulfuric acid and ACH in the first reaction stage (process step a), in a molar ratio of sulfuric acid to ACH in the range from 1.2 to 2; preferably 1.25 to 1.6; more preferably of 1.4 to 1.45. Preference is given to using two or more reactors I in the first reaction stage, in which case sulfuric acid and ACH are used in the first reactor I (reactor A) in a molar ratio of sulfuric acid to ACH in the range from 1.6 to 3; preferably 1.7 to 2.6; more preferably 1.8 to 2.3; and wherein sulfuric acid and ACH are used in the last reactor I (for example in the second reactor I, reactor D) in a molar ratio of sulfuric acid to ACH in the range from 1.2 to 2.0; preferably from 1.2 to 1.8; especially preferably from 1.3 to 1.7. An optimal compromise between achievable yield and sulfuric acid consumption is found to be a molar ratio of 1.25 to 1.6, which can ultimately be assessed from the standpoint of economic optimization. More sulfuric acid increases the costs for this feedstock and the expenditure required for the disposal of the resulting waste acid mixture, but the yield based on ACH can also be increased slightly once again.

The reaction of acetone cyanohydrin with sulfuric acid in the first reaction stage is exothermic. It is therefore advantageous to largely or at least partly remove the heat of reaction obtained, for example with the aid of suitable heat exchangers, in order to obtain an improved yield. Since the viscosity of the reaction mixture rises significantly with falling temperature, and hence circulation, flow and heat exchange in the reactors I (reactor A or D) are limited, excessive cooling should generally be avoided, however. Furthermore, there can be partial or complete crystallization of ingredients on the heat exchangers at low temperatures in the first reaction mixture, which can lead to abrasion, for example in the pump housings, pipelines and heat exchanger tubes of the reactors 1. If the temperature goes too far below the permissible amidation temperature, so as to result in salt formation and precipitation, this often leads to unwanted shutdown of the plant.

For cooling of the reactor circuits, it is possible in principle to use known and suitable cooling media. It is advantageous to use cooling water. Typically, the cooling medium, especially the cooling water, has a temperature below the process conditions chosen. Advantageously, the cooling medium, especially the cooling water, has a temperature in the range from 20 to 90° C., preferably from 50 to 90° C. and more preferably from 60 to 70° C.

To stop temperature from going below the crystallization point of methacrylamide, the heat exchanger is typically operated with a hot water secondary circuit. Preference is given here to temperature differences in the inlet/outlet of the apparatus on the product side of about 1 to 20° C., especially 2 to 7° C.

The conversion of ACH and sulfuric acid in one or more reactors I in a first reaction stage (amidation) is effected at a temperature in the range from 70 to 130° C., preferably 70 to 120° C., more preferably from 85 to 110° C. If two or more reactors are used for the amidation, the temperatures of the various reactors may be the same or different. With regard to different stoichiometries in the reactors, it is preferable to operate the first reactor having a higher sulfuric acid excess at a lower temperature than the downstream reactor(s). The amidation in the first reaction stage in the reactor I or in multiple reactors 1 is often conducted at standard pressure.

Typically, the first reaction stage (amidation) can be performed batchwise and/or continuously. The first reaction stage is preferably conducted continuously, for example in one or more loop reactors. Suitable reactors and processes are described, for example, in WO 2013/143812. Advantageously, the first reaction stage can be conducted in a cascade of two or more loop reactors. Especially preferably, the conversion in the first reaction stage is effected in one or more (preferably two) loop reactors, where the reaction mixture(s) are reacted at a circulation ratio (ratio of circulation volume flow rate to feed volume flow rate) in the range from 5 to 90, preferably 10 to 70. Alternatively, it is possible to use stirred or circulation-pumped continuous stirred tank reactors (CSTRs), or a combination of CSTR apparatuses and loop reactors.

The dwell time in the amidation step is designed such that the time is sufficient to maximize the yield of HIBAm, SIBA, MA and MAA. Typically, the static dwell time in the reactors I, especially in the loop reactors I, is in the range from 5 to 35 minutes, preferably from 8 to 20 minutes.

A suitable loop reactor preferably has the following elements: one or more addition points for ACH, one or more addition points for sulfuric acid, one or more gas separators, one or more heat exchangers, one or more mixers, and a pump. The mixers are frequently executed as static mixers.

The ACH can be added in principle at any point to the one or more reactors I (e.g. loop reactors). However, it has been found to be advantageous when the ACH is added at a well-mixed site. Preference is given to adding the ACH to a mixing element, for example to a mixer having moving parts, or to a static mixer.

The sulfuric acid can be added (2) in principle at any point to the one or more reactors I (e.g. loop reactors). The sulfuric acid is preferably added upstream of the addition of the ACH. Particular preference is given to adding the sulfuric acid on the suction side of the respective reactor pump. It is often possible thereby to improve the pumpability of the gas-containing reaction mixture.

The reactors I (e.g. loop reactors 1) preferably each include at least one gas separator. Typically, it is possible to withdraw product stream (first reaction mixture) continuously via the gas separator on the one hand; on the other hand, it is possible to remove and discharge gaseous by-products. Typically, the gaseous by-product formed is mainly carbon monoxide. The offgas stream from the reactors I is preferably combined with the offgas from the buffer vessel/separator I.

In a preferred embodiment, the first reaction stage comprises the reaction of acetone cyanohydrin (ACH) and sulfuric acid in at least two separate reaction zones, preferably in at least two separate reactors, preferably in at least two loop reactors (e.g. (A) and (D)).

Preference is given to reacting acetone cyanohydrin (ACH) and sulfuric acid in such a way that the reaction volume is divided into at least two reaction zones, and the total amount of ACH is metered separately into the different reaction zones. The amount of ACH which is supplied to the first reactor or to the first reaction zone (e.g. (1a)) is preferably not less than the amounts of ACH that are supplied to the downstream reactors or to the downstream reaction zones (e.g. (1b)).

Preference is given to introducing 50-90% by weight, preferably 60% to 75% by weight, of the total volume flow rate of ACH supplied into the first reactor (e.g. (1a)). The remaining amount of ACH supplied is introduced into the second reactor and optionally into further reactors (e.g. (1b)). Typically, the total amount of ACH is divided between the first reactor I (e.g. (A)) and the second reactor I (e.g. (B)) in a mass ratio of first reactor I:second reactor I in the range from 70:30 to 80:20, preferably of about 75:25.

The molar ratio of added sulfuric acid to ACH in the first reactor or in the first reaction zone is preferably greater than the corresponding molar ratio in the downstream reactors or in the downstream reaction zones.

Especially preferably, the first reaction stage comprises the reaction of (ACH) and sulfuric acid in at least two separate reactors, preferably at least two loop reactors, wherein sulfuric acid and ACH are used in the first reactor in a molar ratio of sulfuric acid to ACH in the range from 1.6 to 3.0, preferably 1.8 to 3.0, and wherein sulfuric acid and ACH are used in the second reactor in a molar ratio of sulfuric acid to ACH in the range from 1.2 to 2.0, preferably from 1.3 to 1.7.

In a particularly preferred embodiment, the conversion in the first reaction stage is effected in two or more loop reactors (e.g. (A) and (D)), in which case the total amount of ACH is metered into the first and at least one further loop reactor. Especially preferably, each loop reactor comprises at least one pump, a heat exchanger cooled with water as medium, a gas separation apparatus, at least one offgas conduit connected to the gas separation apparatus, and at least one feed conduit for ACH in liquid form. Preferably, the at least two loop reactors are connected to one another in such a way that the entire resulting reaction mixture from the first reactor is guided into the downstream reactors, and the reaction mixture in the downstream reactors is admixed with further liquid ACH and optionally further amounts of sulfuric acid.

The first loop reactor is typically operated at a circulation ratio (ratio of circulation volume flow rate to feed volume flow rate) in the range from 5 to 110, preferably 10 to 90, more preferably 10 to 70. In a subsequent loop reactor, the circulation ratio is preferably within a range from 5 to 100, preferably from 10 to 90, more preferably from 10 to 70.

Typically, after the first reaction stage (amidation), a first reaction mixture (6) is obtained, containing 5% to 35% by weight of sulfoxyisobutyramide (SIBA), 5% to 25% by weight of methacrylamide (MAA) and <5% hydroxyisobutyramide (HIBAm), based in each case on the overall reaction mixture, dissolved in the sulfuric acid reaction matrix. This first reaction mixture is preferably conveyed into the second reaction stage with the aid of a discharge pump (E) at a constantly controlled mass flow rate. The constantly controlled mass flow rate especially enables exact control over the static dwell time of the reaction mixture in the second reaction stage (process step (b), conversion).

Preferably, the first reaction mixture resulting from the first reaction stage, proceeding from at least one reactor I, is conveyed through at least one reactor II at a constantly controlled mass flow rate by means of a discharge pump.

Second Reaction Stage (Conversion)

The process according to the invention comprises, in step (b), the converting of the first reaction mixture, comprising heating to a temperature in the range from 130 to 200° C., preferably 130 to 170° C., more preferably 140 to 170° C., further preferably 140 to 165° C., in one or more reactors II (especially also referred to as thermal conversion apparatus or conversion reactors) in a second reaction stage (conversion) to obtain a second reaction mixture (e.g. (7a)) comprising predominantly methacrylamide (MAA) and sulfuric acid.

According to the invention, the conversion comprises the dwell time of the reaction mixture in the second reaction stage being in the range from 2 to 15 min, preferably from 2 to 10 min. Typically, the dwell time specified is based on the overall conversion reactor (reactor II). Typically, the dwell times in all conversion reactors are within the ranges specified. Preferably, the dwell time in the preheater segment of the conversion reactor is 0.1 to 5 min; more preferably 0.2 to 2 min.

According to the invention, the conversion comprises performing the heating in the second reaction stage in one or more reactors II, wherein at least one reactor Il comprises a preheater segment comprising one or more linear pipeline elements that are heated by means of a heating medium spatially separate from the reaction mixture, wherein the reaction mixture is heated by 10 to 100° C., preferably by 15 to 80° C. Typically, heating by 10 to 100° C. should be understood such that a corresponding increase in temperature takes place proceeding from the temperature of the reaction mixture that enters the respective preheater segment of the conversion reactor (reactor II).

According to the invention, the conversion comprises performing the converting in the second reaction stage in one or more reactors II, wherein at least one reactor II comprises at least one delay segment which is operated under virtually adiabatic conditions. Typically, the at least one delay segment comprises pipeline elements that are not trace-heated with heating medium and in which the reaction mixture is thus especially guided in a virtually adiabatic manner.

According to the invention, the converting comprises implementing the preheater segment and/or the delay segment of the conversion reactor (reactor II) in a combination of linear pipeline elements and deflections, wherein the linear pipeline elements and the deflections are connected to one another via reducing flanges. Typically, a reducing flange is a pipe element having an internal cross section that runs convergently (narrows) in flow direction.

Preferably, the at least one conversion reactor (reactor II) comprises at least one preheater segment and at least one delay segment. Especially preferably, all conversion reactors (reactors II) comprise at least one preheater segment and at least one delay segment.

More preferably, the reaction mixture (e.g. (3)) leaving a first reactor I (e.g. reactor A) is already subjected to such heating (conversion) prior to entry into the further loop reactor(s) I (e.g. reactor D). This embodiment is also referred to hereinafter as intermediate conversion.

Typically, during the conversion, in the course of heating of the first reaction mixture to a temperature in the range from 130 to 200° C., the amount of MAA or MAA·H₂SO₄ is increased by dehydrating the HIBAm or by eliminating sulfuric acid from SIBA, wherein the first reaction mixture is a sulfuric acid solution comprising SIBA, HIBAm and MAA, each predominantly in the form of the hydrogensulfates.

Preference is given to heating in the second reaction stage (conversion) over a minimum period of time. In particular, the heating in the second reaction stage (i.e. dwell time in the preheater segment of the conversion reactor) is effected for a period of 0.1 to 14 minutes, preferably 0.1 to 5 minutes, more preferably 0.2 to 2 minutes. Typically, the heating of the reaction mixture as the mixture resulting from the reactors I is conducted for a shorter period than the conversion in the virtually adiabatic elements themselves.

Especially preferably, the conversion is effected at a temperature in the range of 130 to 200° C., preferably 130 to 170° C., more preferably 140 to 170° C., and for a dwell time (i.e. total dwell time in reactor II or reactors II) in the range from 2 to 15 min, preferably from 2 to 10 min.

The conversion can in principle be conducted in known reactors that enable the attainment of the temperatures mentioned within the periods of time mentioned. The energy can be supplied here in a known manner, for example by means of steam, hot water, suitable heat carriers, electrical energy or electromagnetic radiation, such as microwave radiation. Preference is given to conducting the conversion in the second reaction stage in one or more thermal conversion apparatuses.

In a preferred embodiment, the conversion in the second reaction stage is conducted in a thermal conversion apparatus comprising a two-stage or multistage arrangement of pipeline elements. It is possible here to combine linear and curved pipeline elements with one another. Preferably, the multistage tubes are in an opposing arrangement of these linear pipeline elements, connected by curved pipeline elements in order to keep the space demands of the thermal conversion apparatus low.

In the process step of conversion, a high yield of the value-forming components MA and MAA is especially achieved in that the incoming reaction mixture is heated to a defined temperature for a defined period of time. It is advantageous here to heat the reaction mixture very rapidly and within a first defined period of time to the temperature required (preheating, preheater segment) and then to leave it at that temperature for a second defined period of time (dwelling, delay segment). The temperature regime thus preferably includes a first heat-supplying step and a subsequent, virtually adiabatic step in which no heat is supplied from the outside and the temperature of the reaction mixture varies solely as a result of unavoidable heat losses. A virtually adiabatic temperature regime is preferably understood to mean that there is a change in the temperature of the reaction mixture in the delay step (or in the delay segment) by max. 10 K.

Rapid heating in the context of the process according to the invention is typically assured when the first time taken for preheating (dwell time in the preheater segment) is not greater than the second time taken for dwelling (dwell time in the delay segment). Preferably, in the process according to the invention, at least 60% of the average dwell time of the conversion is taken up in the form of the second period (period of time in the delay segment). It is especially crucial for the achievement of a high yield that only very few volume elements of the reaction mixture leaving the conversion reactor have a dwell time that differs from the average dwell time in the conversion reactor. In this context, the person skilled in the art makes reference to a narrow dwell time distribution.

Preferably at least 60% of the dwell time, especially of the static dwell time, is implemented by at least one second reaction stage (conversion) in a delay segment executed as a pipeline.

The control and precise adjustment of a very narrow dwell time distribution in the thermal conversion apparatus (conversion reactor, reactor II) is preferably assured by the maintenance of a plug flow profile and the minimization of backmixing in all pipeline elements and deflections through which the reaction mixture flows. In principle, the thermal conversion apparatus (conversion reactor II) may theoretically be configured as a straight pipe. In a preferred embodiment that takes up less construction space, the thermal conversion apparatus (conversion reactor II) contains linear pipeline elements and deflections. Additional turbulence-generating internals, torsion paddles or guide plates in the interior of the pipeline elements that can be subject to fouling in sustained operation and hence endanger undisrupted operation of the process are preferably not required, by contrast with the prior art (EP 0999200 B1).

In the process according to the invention, the plug flow profile can especially be ensured by means of suitable matching of pipe lengths and diameters, and the dimensions of the deflections, to the reaction mixtures that flow within the apparatuses. More particularly, the process according to the invention ensures that backmixing effects at the transition between linear pipeline elements and deflections and within the deflections are minimized, and hence a spread in the dwell time distribution of heated reaction mixtures is very substantially avoided.

The thermal conversion apparatus used for the conversion, according to the elucidations made, is preferably composed of a first preheater segment and a downstream delay segment. The preheater segment may comprise one or more linear pipeline elements that are heated with a heating medium spatially separate from the reaction mixture that flows through them, wherein the reaction mixture is heated by 10 to 100° C., preferably by 15 to 80° C.

In a preferred embodiment, the superficial volume of the linear pipeline elements relative to the superficial volume of the deflections in the preheater segment and/or in the delay segment corresponds to a ratio (Vₗᵢₙₑₐᵣ/V_{deflection}) in the range from 1.0 to 75.0; preferably in the range from 2.0 to 75.0; preferably in the range from 4.0 to 75.0; especially preferably in the range from 5.0 to 50.0; further preferably in the range from 5.0 to 20.0. Especially preferably, the superficial volume of the linear pipeline elements relative to the superficial volume of the deflections in at least one preheater segment, preferably in all preheater segments, corresponds to a ratio (Vₗᵢₙₑₐᵣ/V_{deflection}) in the range from 1.0 to 75.0; preferably in the range from 2.0 to 75.0; preferably in the range from 4.0 to 75.0; especially preferably in the range from 5.0 to 50.0; further preferably in the range from 5.0 to 20.0.

In a preferred embodiment, at least one preheater segment comprises linear pipeline elements and deflections, wherein the linear pipeline elements of the preheater segment comprise 1 to 50 separate straight pipelines that are secondarily heated with a heating medium and are arranged parallel to one another, and wherein the deflections that connect the linear pipeline elements to one another comprise at least one pipeline.

In a preferred embodiment, in the preheater segment, the linear pipeline elements are executed in one or more straight pipelines having an internal diameter in the range from 8 mm to 200 mm, wherein the pipelines of the linear pipeline elements in the preheater segment may have a smaller internal diameter than the pipelines of the linear pipeline elements in the delay segment.

Preferably, the preheater segment comprises 1 to 50, preferably 2 to 20, separate linear pipeline elements each having an internal diameter of 8 mm to 200 mm, preferably of 12 mm to 160 mm, where preferably all linear pipeline elements have the same diameter.

Flow of the reaction mixture through the linear pipeline elements preferably results in an average flow rate of at least 0.2 m/s.

Preferably, the one or more heated linear pipeline elements of the preheater segment are arranged stacked parallel to one another in a holder or frame in order to keep the space requirement of the preheater segment low. The arrangement is typically configured as a bundle of tubes. It is possible here for the reaction mixture to flow through the multiple linear pipeline elements in parallel (in cocurrent) or in series (alternately in countercurrent), preference being given to series operation. The linear pipeline elements through which the flow passes in series can be connected on the process side by means of known devices, for example domes or deflections.

It has been found that a preferably advantageous construction of the deflections for the achievement of high yields is one in which the deflections are very substantially free of dead spaces and are implemented by means of two pipeline elements each bent by 90° or by means of one pipeline element bent by 180°, and in which the deflection has a single internal cross section. The deflection is preferably implemented by means of two pipeline elements bent by 90° that are connected to one another by a second, preferably unheated, linear pipeline element, wherein the total superficial volume of all linear pipeline elements of the preheater segment relative to the superficial volume of the deflections of the preheater segment corresponds to a ratio in the range from 1.0 to 75.0; preferably in the range from 2.0 to 75.0; preferably in the range from 4.0 to 75.0; especially preferably in the range from 5.0 to 50.0; further preferably in the range from 5.0 to 20.0. More particularly, the superficial volume of the deflection in each case encompasses the volume of the two pipeline elements bent by 90° and the volume of the linear pipeline element in between.

In a preferred embodiment, the linear pipeline elements in the preheater segment and/or in the delay segment of the at least one reactor II are executed in parallel bundles of tubes, and these bundles of tubes are connected by means of at least one deflection, wherein the deflection is configured as a pipeline comprising a first section having an internal cross section that runs convergently in flow direction, a second section having an essentially constant internal cross-sectional area, and a third section having an internal cross section that runs divergently in flow direction. The second section of the deflections that has an essentially constant internal cross-sectional area may preferably be configured as pipe bends, preferably as 90° or 180° pipe bends.

The deflections (for example the deflections in the preheater segment and/or in the delay segment) are preferably formed from pipeline elements having a bend of 90° to 180°. Especially preferably, the deflections are about 180°.

For the purposes of precise control of the dwell time distribution in the overall preheater segment, it is especially advantageous to adjust the internal diameter of the deflections such that the backmixing at the transition between the linear pipeline elements and the deflections and within the deflections themselves is minimized, and a high linear flow rate is maintained. This is preferably ensured by observing a minimum ratio of the cross-sectional area of the linear heated pipeline elements through which reaction mixture flows to the free cross section of the deflections (especially the free cross section of the second section of the deflection that has an essentially constant internal cross-sectional area), (Aₚᵢₚₑ/A_{deflection}), of at least 0.1 m²/m². Preference is given to observing a cross-sectional ratio of 0.8 m²/m², corresponding to a flow rate in the deflections of >0.8 m/s.

The heating medium used may be any heating medium known to the person skilled in the art, for example a thermally durable oil, a salt bath, electromagnetic radiation, an electrical heater, superheated water or steam. Preference is given to using saturated steam as heating medium.

The delay segment, analogously to the preheater segment, may consist of one or more pipes, except that these, by contrast, are unheated. Preferably, the one or more pipes of the delay segment are arranged stacked parallel to one another in a holder or frame in order to keep the space requirement of the delay segment low. The arrangement is likewise typically configured as a bundle of tubes. It is possible here for the reaction mixture to flow through the multiple pipes in parallel (in cocurrent) or in series (alternately in countercurrent), preference being given to series operation. The connection of the tubes through which the flow passes in series on the process side can be executed by any type of connection known to the person skilled in the art; preference is given to using the combination of pipeline elements already described as deflection for the preheater segment. The linear pipeline elements of the delay segment preferably have an internal diameter in the range from 8 mm to 200 mm, where the linear and curved pipeline elements of the delay segment may have a greater internal diameter than the corresponding pipeline elements of the preheater segment.

As has been shown by CFD simulations, from a construction point of view, observance of particular geometries and ratios of dimensions of the curved pipeline elements that form the deflections and of the second linear pipeline elements provided between the deflections is preferable for minimization of backmixing effects, the assurance of a plug flow, and hence for precise adjustment and control of the dwell times in the preheater or delay segment. Compliance with these construction parameters surprisingly also permits the continuance of the advantages mentioned in the case of partial load operation of the process described, i.e. at flow rates lower than in the case of the rated load. It can thus be shown that the yield achieved is similar or identical in the partial load range to the yield achieved at the design point for full load. “Partial load” refers to load conditions of the plant where the feed mixture is generally reduced compared to the mode of operation at full load. A partial load of the plant comprises the reduction of the feed rates by up to 70%, preferably by up to 50%.

For minimization of backmixing effects in the deflections that connect the linear pipeline elements, the internal diameter of the deflections is preferably chosen so as to establish, within the deflections, an average speed of the reaction medium of 0.2 to 3 m/sec, preferably of 0.2 to 2 m/sec, especially preferably of 0.5 to 2 m/sec, and such that the variance in the local speed at any point within the deflections from the average conveying speed is not more than 30%. Preferably, within the deflections, an average speed of the reaction mixture of 0.2 to 3 m/sec, preferably of 0.2 to 2 m/sec, especially preferably of 0.5 to 2 m/sec, is established. Preferably, within the deflections, an average speed of the reaction mixture of 0.2 to 3 m/sec, preferably of 0.2 to 2 m/sec, especially preferably of 0.5 to 2 m/sec, is established, and the variance in the local speed at any point within the deflections from the average conveying speed is not more than 30%.

The construction used for the conversion reactors, comprising at least one preheater segment and at least one delay segment, preferably comprises linear pipeline elements and deflections. The pipelines and pipeline elements of the conversion reactor may preferably consist of any material known to the person skilled in the art that is resistant to strong acids under the reaction conditions and temperatures described. Suitable materials for the preheater segments comprise, but are not limited to, Hastelloy alloys, tantalum alloys, and silicon carbide-containing ceramics. The delay segments may be constructed from less expensive materials such as steel/PTFE combinations, steel/enamel combinations or steel/ceramic combinations. Typically, the preheater segments are constructed from materials of high thermal conductivity, while the materials of the delay segments feature a lower thermal conductivity.

In a preferred embodiment, reducing flanges are installed on at least one shell-and-tube heat exchanger at the transition from the linear pipeline elements to the deflections, and these are especially executed as reducing flanges having a low level of dead space, so as to achieve an area ratio between the free cross-sectional area of the linear heated pipeline elements through which reaction mixture flows to the free cross section of the deflections, (A_{tube}/A_{deflection}), of at least 0.1 m²/m². In particular, the free cross-sectional area of the linear heated pipeline elements is based on the sum total of the free cross-sectional areas of all pipelines of the linear pipelines (or pipeline bundles).

In a preferred embodiment, a 180° pipe bend is installed in at least one of the reducing flanges, having a ratio of the free cross-sectional area of the linear pipeline elements present within the heat exchanger to the free cross-sectional area of the deflections of at least 0.8 m²/m² and hence achieving at least a flow rate of >0.8 m/s within the deflections. The dwell time in the second reaction stage (process step (b), conversion), comprising the dwell time in the preheater segment and the dwell time in the delay segment, may especially vary depending on the reaction mixtures and temperatures. It has been found that, for completion of the desired reactions (HIBAm→MAA and SIBA→MAA) on the one hand and for minimization of the further reactions that reduce the yield on the other hand, a defined dwell time range is advantageous. This dwell time range is typically 2 to 15 minutes.

More particularly, the conversion reactor is configured so as to take account of the following factors: maintenance of plug flow, the desired dwell time, the flow rate, the reaction mixture and the temperature regime within the converters.

In a preferred embodiment (intermediate conversion), at least one intermediate conversion between at least two first reaction stages (amidation) takes place in the performance of the first reaction stage (amidation) and the second reaction stage (conversion), wherein the at least two reactors II for the conversion are implemented by at least one shell-and-tube heat exchanger as preheater segment, combined with at least one delay zone as delay segment.

In a preferred embodiment (intermediate conversion), at least two reactors I (amidation) are used, wherein at least one reactor II comprising at least one preheater segment and/or delay segment between the reactors I, and at least one reactor II comprising at least one preheater segment and/or delay segment at the exit from the first reaction stage (amidation) are used.

In the embodiment with intermediate conversion, it is possible to use an intermediate cooler in order to cool the hot conversion mixture before it is returned to the amidation. But the cooler can be dispensed with if the cooling apparatuses within the amidation are of sufficient dimensions to reduce the heat of conversion.

Preferably, at least one preheater segment in at least one reactor II is configured as a shell-and-tube heat exchanger, wherein the reaction mixture flows through the shell-and-tube heat exchanger on the tube side, and the free cross-sectional area of the tubes used in the shell-and-tube heat exchanger ensures an average flow rate of at least 0.2 m/s in the tubes.

The thermal conversion apparatus used for the conversion may further preferably be combined with one or more gas separators. For example, it is possible to guide the reaction mixture through a gas separator after it leaves the preheater segment of the thermal conversion apparatus (reactor II) and/or after it leaves the delay segment of the thermal conversion apparatus (reactor II). It is especially possible here to separate gaseous by-products from the reaction mixture.

In a preferred embodiment, the second reaction mixture obtained in step b, comprising predominantly methacrylamide and sulfuric acid, is cooled down to a temperature below 120° C., preferably to a temperature in the range from 90 to 120° C., preferably to a temperature in the range from 90 to 119° C., for example in a cooler with a cooling medium having a temperature in the range from 60 to 100° C. Preferably, the second reaction mixture obtained in step b, preferably after cooling, is intermediately buffered in an intermediate vessel (also referred to as buffer vessel, e.g. (1)) before the reaction mixture is guided into the third reaction stage. The dwell time in the intermediate vessel (buffer vessel) (e.g. (1)) is preferably in the range from 5 to 60 min.

Typically, the first reaction mixture obtained in the first reaction stage is guided completely into reactor II of the second reaction stage. Preferably, downstream of each reactor 1 executed as a loop reactor, the exiting first reaction mixture is guided into a reactor II executed as a thermal conversion apparatus. The second reaction mixture exiting from such a reactor II can itself again be guided into a further, downstream reactor I or cooled in a cooler (e.g. (H)) and then stored in a buffer vessel (e.g. (1)) before being sent as a stream (e.g. (8)) to the third reaction stage (c) (esterification or hydrolysis).

The still-hot reaction mixture exiting from the conversion (e.g. (7a)), prior to entry into the buffer vessel (e.g. (1)), is preferably cooled in a cooler (e.g. (H)) to obtain a cooled stream (e.g. (7b)) in order to assure a defined dwell time of the reaction mixture leaving the last conversion reactor at high temperatures and to suppress yield-reducing further reactions of the MA and MAA products of value formed in the conversion, and wherein gaseous by-products may be at least partly separated from the second reaction mixture. The buffer vessel (e.g. (I)) firstly fulfils the function of ensuring a uniform feed stream to the downstream third reaction stage (esterification or hydrolysis). Secondly, it especially permits further degassing of the cooled second reaction mixture and hence functions as a gas separator. Typically, the degassed second reaction mixture is guided fully into the third reaction stage (esterification or hydrolysis). Preferably, the offgas (9) which is obtained in the buffer vessel (e.g. (1)) after the conversion is discharged fully or partly from the process. Further preferably, the offgas which is obtained after the conversion in the gas separator is guided fully or partly into the third reaction stage (esterification or hydrolysis).

The cooler (e.g. (H)) may preferably be executed as at least one heat exchanger which is used to lower the temperature of the reaction mixture (e.g. (7a)) before the esterfication reaction and to prevent degradation of the products of value. The cooler (e.g. (H)) may typically be executed as any type of heat exchanger known to the person skilled in the art that is stable at the temperature described and under the strongly acidic conditions. Suitable heat exchangers include plate and frame, plate and fin, spiral and tube. Suitable construction materials may include, but are not limited to, Hastelloy B, Hastelloy B-2, Hastelloy B-3, silicon carbide and tantalum alloys, especially also materials and material combinations containing enamel, other ceramic materials, and chemically and thermally stable plastics.

For cooling of the second reaction mixture, it is possible in principle to use known and suitable cooling media. It is advantageous to use cooling water. Typically, the cooling medium, especially the cooling water, has a temperature below the process conditions chosen. Advantageously, the cooling medium, especially the cooling water, has a temperature in the range from 30 to 120° C., preferably from 50 to 110° C. and more preferably from 60 to 100° C. The exit temperature of the cooled reaction mixture (e.g. (7b)) leaving the cooler (e.g. (H)) and hence the inlet temperature into the downstream reaction step (third reaction stage (esterification or hydrolysis) (c)) is preferably below the maximum temperature that occurs in the conversion reactor, typically in the range from 70° C. to 110° C.

Third Reaction Stage (Esterification or Hydrolysis)

The process according to the invention comprises, in step (c), the reacting of the second reaction mixture comprising predominantly MAA with water and optionally methanol in one or more reactors Ill in a third reaction stage (esterification or hydrolysis) to obtain a third reaction mixture comprising methacrylic acid and/or methyl methacrylate.

In a preferred embodiment (esterification), the second reaction mixture comprising predominantly MAA is reacted with water and methanol to obtain a third reaction mixture comprising methyl methacrylate. The conditions for the esterification on an industrial scale are known to the person skilled in the art and are described, for example, in U.S. Pat. No. 5,393,918.

In a further embodiment (hydrolysis), the second reaction mixture comprising predominantly MAA is reacted with water to obtain a third reaction mixture comprising methacrylic acid. The conditions of the hydrolysis of methylacrylamide to methacrylic acid and the processes for workup on an industrial scale are known to the person skilled in the art and are described, for example, in DE 10 2008 000 787 A1 and EP 2 292 580.

The conversion in the third reaction stage (esterification or hydrolysis) is preferably conducted in one or more suitable reactors, for example in heated tanks. In particular, it is possible to use steam-heated tanks. In a preferred embodiment, the esterification is effected in two or more, for example three or four, successive tanks (tank cascade).

Typically, the esterification is conducted at temperatures in the range from 100 to 180° C., preferably from 100 to 150° C., at pressures up to 7 bara, preferably at pressures up to 2 bara, and using sulfuric acid as catalyst.

The second reaction mixture is preferably reacted with an excess of methanol and water. The addition of the second reaction mixture comprising predominantly methacrylamide and the addition of alcohol are preferably effected in such a way as to result in a molar ratio of methacrylamide to alcohol in the range from 1:1.0 to 1:1.6. In a preferred embodiment, the reaction in the third reaction stage is effected in two or more reactors III, in which case there is a molar ratio of methacrylamide to alcohol in the first reactor III in the range from 1:0.7 to 1:1.4 (preferably 1:1.0 to 1:1.4), and in which case there is a molar ratio of methacrylamide to alcohol in the second and possible downstream reactors III in the range from 1:1.05 to 1:1.3.

Preferably, the alcohol supplied to the third reaction stage (esterification or hydrolysis) is composed of alcohol freshly supplied to the process (fresh alcohol) and of alcohol present in recycled streams (recycling streams) in the process according to the invention. It is additionally possible in the process according to the invention to use methanol present in recycling streams from downstream processes.

Typically, water is added to the reactor III or to the reactors III of the third reaction stage in such a way that the concentration of water is in the range from 10% to 30% by weight, preferably 15% to 25% by weight, based in each case on the overall reaction mixture in the reactor III.

In principle, the water supplied to the third reaction stage (esterification or hydrolysis) may come from any source and may contain various organic compounds, provided that no compounds are present that have an adverse effect on the esterification or the downstream process stages. The water supplied to the third reaction stage preferably comes from recycled streams (recycling streams) in the process according to the invention, for example from the purification of the methyl methacrylate or the methacrylic acid. It is additionally possible to supply fresh water, especially demineralized water or well water, to the third reaction stage (esterification or hydrolysis).

Esterification with methanol typically affords a third reaction mixture comprising methyl methacrylate (MMA), methyl hydroxyisobutyrate (MHIB) and further above-described by-products, and also significant amounts of water and unconverted methanol.

In a preferred embodiment, the esterification is effected in two or more (especially three or four) successive tanks (tank cascade), wherein the liquid overflow and the gaseous products are guided from the first tank into the second tank. The corresponding procedure is typically followed with possible downstream tanks. More particularly, such a mode of operation can reduce foam formation in the tanks. In the second tank and in the possible downstream tanks, it is likewise possible to add alcohol. The amount of alcohol added here is preferably at least 10% less compared to the preceding tank. The concentrations of water in the various tanks may typically be different. The temperature of the second reaction mixture fed into the first tank is typically in the range from 100 to 180° C. The temperature in the first tank is typically in the range from 90 to 180° C., and the temperature in the second and in the possible downstream tanks in the range from 100 to 150° C.

Typically, the hydrolysis is effected by reacting the second reaction mixture with water, with hydrolysis of MAA to MA, obtaining the resultant ammonia in the form of ammonium hydrogensulfate. A multitude of such processes is described in the prior art, for example in U.S. Pat. No. 7,253,307. For example, MAA is reacted with water at moderate pressure and temperatures between 50° and 210° in the presence of superstoichiometric amounts of sulfuric acid in stirred tank reactors or circulation reactors to give methacrylic acid.

Typically, the reaction of the second reaction mixture with water (hydrolysis) is effected batchwise or continuously, for example in a tubular reactor or a stirred tank reactor. The reaction of the second reaction mixture with water (hydrolysis) can be performed, for example, in one or more suitable reactors III, for example in heated tanks or tubular reactors at temperatures in the range from 90 to 130° C., preferably from 100 to 125° C. The second reaction mixture is preferably reacted with an excess of water in such a way as to result in a molar ratio of water to methacrylamide in the range from 1 to 8, preferably 3 to 6.

The hydrolysis with water typically affords a third reaction mixture comprising methacrylic acid, hydroxyisobutyric acid (HIBAc) and further above-described by-products, and significant amounts of water.

Workup

Typically, the process comprises the separation of methyl methacrylate and/or methacrylic acid from the third reaction mixture (workup) obtained from the third reaction stage (esterification or hydrolysis). It is especially possible here to use the separation steps known to the person skilled in the art, especially rectification, extraction, stripping and/or phase separation steps. The separation of methyl methacrylate and/or methacrylic acid from the third reaction mixture preferably comprises at least one distillation step, preferably at least two distillation steps.

The workup preferably comprises the prepurification of the third reaction mixture which is obtained in the esterification. More particularly, the prepurification comprises at least one distillation step, at least one phase separation step and at least one extraction step. The prepurification preferably comprises at least one, preferably at least two, distillation step(s). More particularly, the prepurification affords a crude MMA product and/or crude MA product having a purity in the region of at least 85% by weight.

Preferably, the workup comprises the fine purification of the crude product, where the fine purification comprises at least one, preferably at least two, distillation step(s). More particularly, the fine purification affords a crude MMA product and/or crude MA product having a purity of at least 99.0% by weight.

In a further embodiment, the workup comprises the separation of methacrylic acid (MA) from the third reaction mixture. Details of the workup for obtaining MA are described in DE 10 2008 000 787 A1.

In a preferred embodiment, the evaporable portion of the third reaction mixture, especially the esterification with methanol, which is obtained in the third reaction stage is removed from the reactors III in gaseous form (vapour) and sent to further workup, for example a distillation step. If, preferably, a cascade consisting of multiple reactors III, for example multiple tanks, is used, it is possible to remove the evaporable portion of the resultant reaction mixture as a vapour stream in each tank and guide it to further workup. Preferably, only the evaporable portion of the reaction mixture formed in the last two tanks (as the third reaction mixture) is removed as vapour stream and guided to further workup.

The workup of the third reaction mixtures comprises, for example, a suitable sequence of separating operations, preferably selected from rectification, extraction, stripping and/or phase separation steps. Preference is given to recycling process streams that still contain intermediates convertible to the target product, such as MHIB or HIBAc, into the upstream process steps. Moreover, it should preferably be ensured through suitable choice of the operating parameters of the separating operations that methyl methacrylate already formed does not break down into by-products in a value-reducing manner.

The separation and recycling of these intermediates are associated with energy expenditure, which can be minimized in that the upstream process steps produce minimum proportions of these intermediates. Thus, maximum conversion, especially of HIBAc to MA, in the conversion (process step (b)) generally has an advantageous effect on the overall production process.

It is advantageously possible to add one or more stabilizers in various streams of the process according to the invention in order to prevent or reduce polymerization of the methyl methacrylate and/or of the methacrylic acid. For example, it is possible to add a stabilizer to the third reaction mixture obtained after the esterification. Preference is given to using phenothiazine and other equivalent stabilizers in the amidation and conversion section, and to using phenolic compounds, quinones and catechols in the esterification/workup section. Additionally used are amine N-oxides, for example TEMPOL, or combinations of the stabilizer systems mentioned.

A waste stream consisting essentially of dilute sulfuric acid is preferably removed from the third reaction stage (esterification or hydrolysis). This waste stream is typically discharged from the process. This waste stream, especially together with one or more aqueous waste streams from the process according to the invention, is preferably sent to a process for regeneration of sulfuric acid or a process for obtaining ammonium sulfate.

In a preferred embodiment, the process according to the invention comprises, in the third reaction stage (process step (c)), the esterification of MAA to MMA. Therefore, maximum efficiency of conversion of HIBAm and SIBA to MAA is crucial for a high yield of the overall process. The efficiency of the process according to the invention can thus be ascertained as the difference between the yield after the process step of amidation and the yield after the process step of conversion. The yield after the process step of amidation is found from the measured amounts of HIBAm, SIBA, MA and MAA. The yield after the thermal conversion is found from the measured amounts of MA and MAA.

The process according to the invention, especially the described flow-optimized construction of the reactor used in the conversion step (especially thermal conversion apparatus), allows significant improvement in the overall yield of MMA and/or MA based on ACH used.

Typically, the amidation yield can reach values up to 97% based on the ACH starting material used. The yields of the conversion reaction can preferably be improved by 0.4% to 1% relative to the prior art, based on the conversion of the intermediates present after amidation and the MAA already preformed in the amidation.

The economic importance of the invention described is illustrated in typical production volumes from ACH-based MMA preparation processes. For a typical MMA production plant with a capacity of 250 Id/a, the process improvement described results in an extra production volume of 1000 to 2500 t/a.

Apparatus for Performance of the Process According to the Invention

The present invention additionally relates to an apparatus for performance of the process according to the invention as described above, wherein the apparatus in each case comprises one or more reactors I (amidation reactor), II (conversion reactor) and III (esterification/hydrolysis reactor) that are connected to one another by pipeline elements, and wherein at least one reactor II comprises at least one preheater segment and at least one delay segment, wherein the preheater segment comprises one or more linear pipeline elements that are heated by means of a heating medium spatially separate from the reaction mixture, and wherein the delay segment is operated under virtually adiabatic conditions, and wherein the preheater segment and/or the delay segment are implemented in a combination of linear pipeline elements and deflections, wherein the linear pipeline elements and the deflections are connected to one another via reducing flanges.

The preferred embodiments described above in connection with the process according to the invention are also analogously applicable to the apparatus.

The superficial volume of the linear pipeline elements relative to the superficial volume of the deflections in the preheater segment and/or in the delay segment preferably corresponds to a ratio in the range from 1.0 to 75.0: preferably in the range from 2.0 to 75.0; preferably in the range from 4.0 to 75.0; especially preferably in the range from 5.0 to 50.0; further preferably in the range from 5.0 to 20.0.

Preferably, in the preheater segment, the linear pipeline elements are executed in one or more straight pipelines having an internal diameter in the range from 8 mm to 200 mm, preferably 12 mm to 160 mm, wherein the pipelines of the linear pipeline elements in the preheater segment have a smaller internal diameter than the pipelines of the linear pipeline elements in the delay segment.

Preferably, the deflections are formed from pipeline elements having a bend of 90° to 180°.

In a preferred embodiment, the linear pipeline elements of the preheater segment and/or of the delay segment are connected by deflections, wherein the deflections are an element that causes deflection by 180°, formed from two 90° pipeline deflections and one linear pipeline element preferably disposed between the 90° pipeline deflections.

The invention, and especially the effect of the flow-optimized construction of the thermal conversion apparatus used for the process step of conversion, is illustrated by the examples that follow.

### DESCRIPTION OF THE FIGURES

FIG. 1 describes the reaction network of the formation of methacrylic acid and/or methyl methacrylate proceeding from methane and ammonia, and acetone. Proceeding from methane (CH₄) and ammonia (NH₃), it is possible to prepare hydrogen cyanide via the BMA process (hydrogen cyanide from methane and ammonia) by means of catalytic dehydrogenation (CH₄+NH₃→HCN+3H₂) (variant 1). Alternatively, it is possible to prepare hydrogen cyanide via the Andrussow process proceeding from methane and ammonia, with addition of oxygen (CH₄+NH₃+1.5O₂→HCN+3H₂O) (variant 2). Further sources known to the person skilled in the art for hydrogen cyanide for the preparation of acetone cyanohydrin are acrylonitrile processes, wherein the propylene ammoxidation also forms 5-15% hydrogen cyanide, relative to the main acrylonitrile product, and also what is called the formamide process, wherein formamide is converted in the gas phase under dehydration to HCN.

In the next step, proceeding from acetone and hydrogen cyanide, acetone cyanohydrin (ACH) is prepared with addition of a basic catalyst (e.g. diethylamine Et₂NH or alkali metal hydroxides). The hydroxyl group of acetone cyanohydrin is subsequently esterified with sulfuric acid, initially giving sulfoxyisobutyronitrile (SIBN). The nitrile group of sulfoxyisobutyronitrile (SIBN) can be hydrolysed in the next step under the action of sulfuric acid and water, giving sulfoxyisobutyramide hydrogensulfate (SIBA·H₂SO₄). A side reaction that can proceed is the formation of methacrylonitrile (MAN) with elimination of sulfuric acid from SIBN. Sulfoxyisobutyramide hydrogensulfate (SIBA·H₂SO₄) can additionally be partly hydrolysed to give alpha-hydroxyisobutyramide hydrogensulfate (HIBAm·H₂SO₄). Likewise possible is the reverse reaction to give the sulfuric ester SIBA·H₂SO₄. A by-product formed may be alpha-hydroxyisobutyric acid (HIBAc) via further hydrolysis of HIBAm·H₂SO₄. Proceeding from SIBA·H₂SO₄, with the elimination of sulfuric acid, methacrylamide hydrogensulfate (MAA·H₂SO₄) is formed (conversion). The gradual reaction of HIBAm or HIBAc to give MA or MAA can likewise proceed as an elimination reaction with elimination of NH₄HSO₄ or water. Methacrylamide hydrogensulfate (MAA·H₂SO₄) can subsequently be converted by hydrolysis to methacrylic acid (MA) or by esterification with methanol (MeOH) to methyl methacrylate (MMA). If alpha-hydroxyisobutyric acid (HIBAc) is entrained into the esterification, it can be converted to methyl alpha-hydroxyisobutyrate (MHIB); the reaction of HIBAm with methanol leads to the same reaction product.

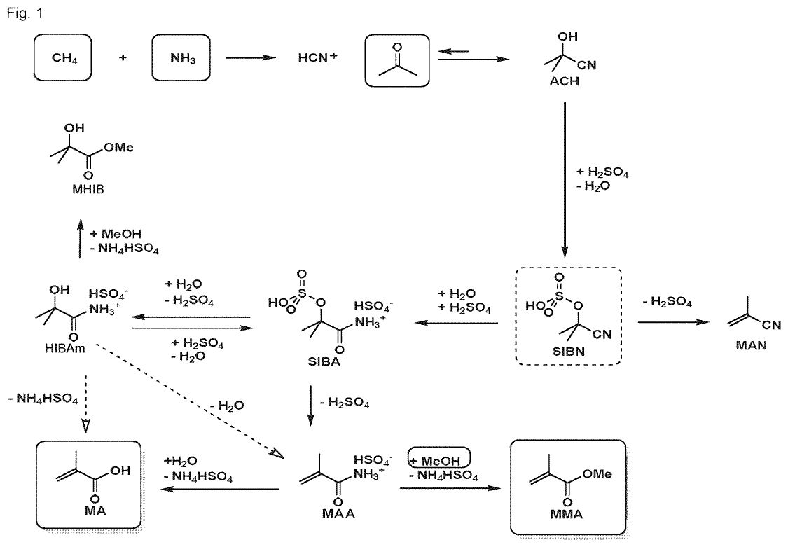

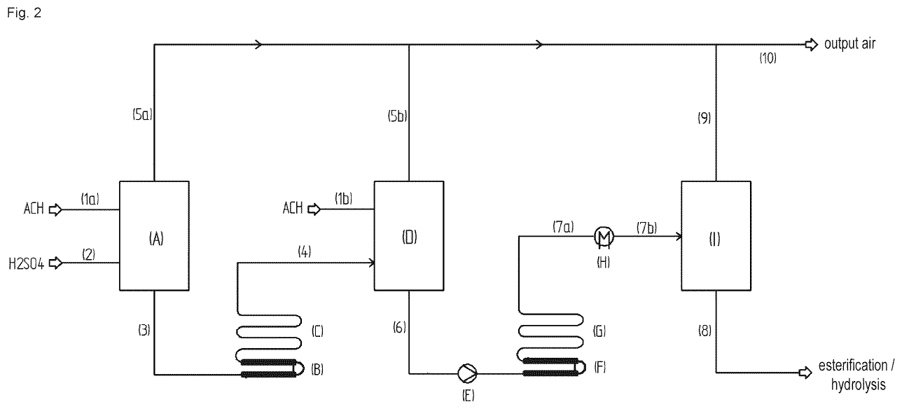

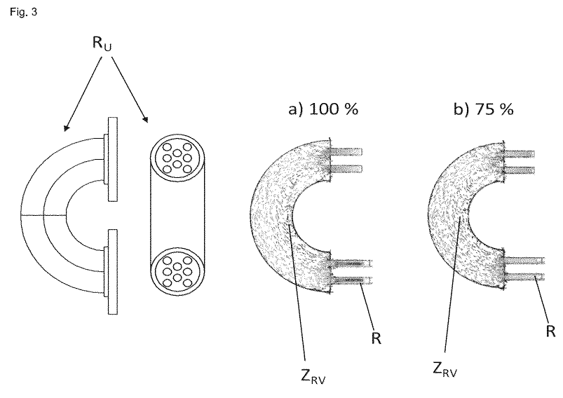

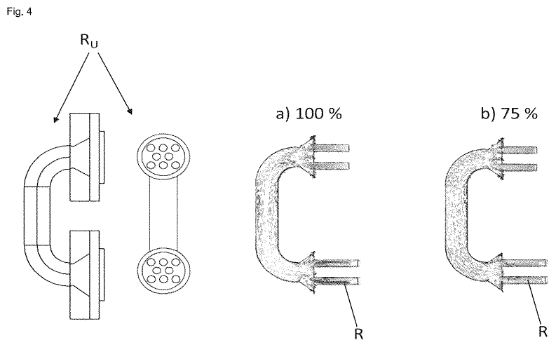

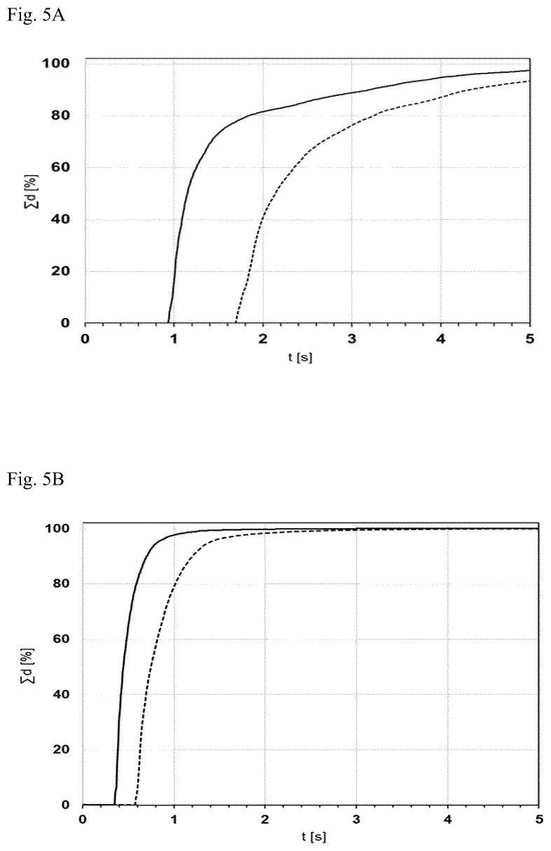

The abbreviations in FIG. 1 and the description for FIG. 1 have the following meanings:
ACH acetone cyanohydrin;
SIBN alpha-sulfoxyisobutyronitrile;
SIBA alpha-sulfoxyisobutyramide;
SIBA·H₂SO₄ alpha-sulfoxyisobutyramide hydrogensulfate;
MAN methacrylonitrile:
HIBAm alpha-hydroxyisobutyramide;
HIBAm·H₂SO₄ alpha-hydroxyisobutyramide hydrogensulfate;
MAA methacrylamide;
MAA·H₂SO₄ methacrylamide hydrogensulfate;
MA methacrylic acid:
MMA methyl methacrylate:
HIBAc alpha-hydroxyisobutyric acid;
MHIB methyl alpha-hydroxyisobutyrate.

FIG. 2 shows a flow diagram of a preferred embodiment of the first and second reaction stages (amidation and conversion) in the process according to the invention. In FIG. 1 are two successive process steps of amidation and conversion comprising the first amidation (A) and the first conversion ((B), (C)), and the second amidation (D) and the second conversion ((F), (G)).

In FIG. 2, the reference symbols have the following meanings:

### Apparatuses:

(A) amidation reactor 1
(B) preheater segment 1
(C) delay segment 1
(D) amidation reactor 2
(E) discharge pump
(F) preheater segment 2
(G) delay segment 2
(H) cooler
(I) buffer vessel

### Streams of Matter:

(1a) acetone cyanohydrin feed to reactor 1
(1b) acetone cyanohydrin feed to reactor 2
(2) sulfuric acid feed to reactor 1
(3) discharge from amidation reactor 1 (liquid)
(4) reaction mixture after intermediate conversion
(5a) offgas from amidation reactor 1
(5b) offgas from amidation reactor 2
(6) discharge from amidation reactor 2 (liquid)
(7a) reaction mixture after conversion
(7b) cooled reaction mixture
(8) discharge from buffer vessel (liquid)
(9) offgas from buffer vessel
(10) overall offgas FIG. 3 shows a 180° pipe deflection Ru in the preheater segment (B) and/or (F) according to Examples 1 and 2, and the illustration of the flow conditions therein. Shown under a) are the flow conditions at 100% load, and under b) the flow conditions at 75% load. Eight linear pipelines R in each case lead into the pipe deflection Ru. Zones of high backmixing in the pipe deflection are marked by Z_{RV}.

FIG. 4 shows a 180° pipe deflection Ru in the preheater segment (B) and/or (F) according to Inventive Examples 3 to 6, and the representation of the flow conditions therein at a) 100% load (see Examples 3, 4, 5) and b) 75% load (see Example 6). Eight linear pipelines R in each case lead into the pipe deflection Ru.

FIG. 5a shows the dwell time distribution (cumulative function) in a pipe deflection according to comparative examples as per FIG. 3 at 100% load (through-flow) in the solid line and 75% load (through-flow) in the dotted line. FIG. 5b shows the dwell time distribution (cumulative function) in a pipe deflection according to inventive examples as per FIG. 4 at 100% load (through-flow) in the solid line and 75% load (through-flow) in the dotted line. The symbols in these figures mean:
Σd: cumulative distribution (integral of the numerical density distribution of 12 000 volume elements considered);
τ: dwell time

The invention is described further by the examples that follow.

### EXAMPLES

The preparation of methacrylamide in sulfuric acid solution, comprising the reaction of acetone cyanohydrin with sulfuric acid in the amidation, the thermal conversion of the mixture in the conversion and the subsequent cooling thereof, was effected according to the embodiment of FIG. 2.

There follows a description of two comparative examples (Example 1 and Example 2) using a construction of the heating/conversion according to the prior art (as per FIG. 3), and of inventive examples (Examples 3-6) using a flow-optimized construction (as per FIG. 4) of the thermal conversion apparatus used for the process step of conversion.

Samples were taken downstream of the conversion reactor. The concentrations of MAA, MA and HIBAm ascertained after quantification by means of HPLC were used for the mass balance of the process steps of amidation and of conversion. Results and overall yield losses over the individual process steps are shown in Tables 1-7 for Comparative Examples 1 and 2, and Inventive Examples 3-6. In the listing of the measurement results, a measurement error in the HPLC analysis of ±0.2% is disclosed in each case.

The mixture in sulfuric acid obtained from the conversion, comprising MAA, MA and HIBAm, can subsequently be reacted with methanol and water to give methyl methacrylate or hydrolysed with water to give methacrylic acid.

### Example 1 (Comparative Example): Preparation of Methacrylamide in Sulfuric Acid Solution Based on Acetone Cyanohydrin

The general procedure for the process as per the flow diagram of FIG. 2 is described hereinafter. In Example 1 (comparative example), pipe deflections according to the prior art as shown in FIG. 3 were used. The results, which are representative of steady-state operation for 7 days, are shown in Table 1. 11 000 kg/h of acetone cyanohydrin having a typical composition of 99.0% acetone cyanohydrin, 0.3% acetone, 0.4% water and sulfuric acid was divided in a mass ratio of 65/35, so as to obtain a stream (1a) of 7100 kg/h and a stream (1b) of 3900 kg/h.

The water content of the ACH feed stream (1a) and (1b) is calculated from the difference from the ACH content, which is ascertained by means of HPLC.

The water content in the sulfuric acid feed (2) is calculated from the difference from the sulfuric acid content which is ascertained by measuring the density and speed of sound.

Feed stream (1a) was subsequently applied to the first amidation reactor (A). Before it entered the amidation reactor, 50 ppm of phenothiazine (as an example of a stabilizer according to the prior art) was added to the sulfuric acid (2).

The amidation reactor (A) was designed as a loop reactor and was operated at 100° C. Feed stream (1a) was fed continuously and at a temperature of 20° C. to the reactor circuit (A) described.

The amount of sulfuric acid (2) needed for the optimal conversion of the reaction mixture in reactors (A) and (D) that has a concentration of 99.7% (0.3% water) was fed to reactor (A) in a mass ratio to the total amount of acetone cyanohydrin (1a+1b) of 1.62 kgH₂SO₄/kgACH. The feed mass flow rate of sulfuric acid used was 17 800 kg/h. This achieved a sulfuric acid excess (sulfuric acid/ACH ratio) of 2.55 kg/kg in reactor (A).

The resultant stirred-up mixture (3) at 100° C., consisting of sulfoxyisobutyramide (SIBA), methacrylamide (MAA) and hydroxyisobutyramide (HIBAm), dissolved in sulfuric acid, with simultaneous separation of gas in circulation operation, was fed continuously to an intermediate conversion (B, C). The pressure differential required for conveying was implemented by means of the reactor circulation pump of the amidation reactor (A). The resultant offgas (5a) was removed from the process in the direction of the output air (10).

The first conversion reactor (B, C) was executed as a flow tube reactor consisting of a preheater segment (B) and a delay segment (C). The reaction mixture entering the intermediate conversion reactor (B, C) was first divided into two substreams and heated to 130° C. in the preheater segment (B) consisting in each case of 3 series-connected shell-and-tube heat transferrers of nominal width DN150 at the exit from the apparatus on the process side.

The reaction medium was guided exclusively on the tube side within the preheater segment (B), while the respective heat exchanger shell was heated with 12 bar(g) saturated steam. Each heat exchanger was provided with 8 tubes that had an internal diameter of 18 mm, such that a flow rate in the tubes of 1.22 m/s was obtained.

The individual heat transferrers were arranged parallel to one another in a frame as an integrated system, with the connection of the series-connected apparatuses on the process side implemented by means of two pipeline segments each bent by 90° (collectively 180°). Also implemented in the preheater segment (B) was a superficial volume ratio of the linear pipeline sections relative to the bent pipeline sections or relative to the deflections, Vₗᵢₙₑₐᵣ/V_{deflection}, according to Table 7.

The nominal width of the connecting pipeline elements corresponded to that of the shell of the shell-and-tube apparatuses, and was likewise DN150 (internal diameter di=158 mm). This gives an average flow rate of the reaction medium in these sections according to Table 7.

The reaction mixture heated in this way was then combined from the two parallel preheater segments (B) and guided through a virtually adiabatically operated delay segment (C). The delay segment (C) was composed of 12 series-connected linear reactor segments which, as in the preheater segments, were arranged parallel to one another and were connected to one another by 180° pipe bends of equal nominal width.

The tubes of the delay segment, and also the bent connecting pipeline elements, are executed with an internal diameter of d_{I}=158 mm, and hence result in a flow rate of the reaction mixture as shown in Table 7. A superficial volume ratio of the linear pipeline sections relative to the bent pipeline sections or relative to the deflections. Vₗᵢₙₑₐᵣ/V_{deflection}, according to Table 7 was implemented in the delay segment (C). As a result of the volume of the heating segment (B) of the delay segment (C) and of the connecting pipelines, an average static dwell time in the intermediate conversion (B, C) according to Table 7 was achieved.

The reaction mixture (4) exiting from the first conversion reactor (B, C) was subsequently fed to the second amidation reactor (D). The amidation reactor (D) is constructed as a loop reactor analogously to the reactor (A) and was likewise operated at about 100° C. The amount of 3900 kg/h of acetone cyanohydrin (1b) needed for the second reaction step was also added directly to the reaction mixture in the second amidation reactor (D). The resultant mass ratio established in the reaction mixture was found to be 1.62 kg H₂SO₄/kg ACH. The resultant offgas (5b) was removed from the process in the direction of the overall offgas (10).

Withdrawn from the second amidation reactor (D) by means of a discharge pump (E), continuously and under volume flow control, was a reaction mixture (6) at 100° C., consisting of sulfoxyisobutyramide (SIBA), methacrylamide (MAA) and hydroxyisobutyramide (HIBAm) (dissolved in sulfuric acid), which, for final conversion of the reactive constituents, was sent to the second conversion step (F, G).

The second conversion reactor (F, G) was likewise executed as a flow tube reactor consisting of a preheater segment (F) and a delay segment (G). The reaction mixture entering the conversion reactor (F, G) was first divided into two substreams and then heated to 158° C. in the heating segment (F) consisting in each case of 12 series-connected shell-and-tube heat transferrers of nominal width DN150 at the exit from the apparatus on the process side. The reaction medium was guided exclusively on the tube side within the preheater segment (F), while the respective heat exchanger shell was heated with 12 bar(g) saturated steam.

Each heat exchanger was provided with 8 tubes that have an internal diameter of 18 mm, such that a flow rate in the tubes according to Table 7 was obtained. The individual heat transferrers were arranged parallel to one another in a frame as an integrated system, with the connection of the series-connected apparatuses on the process side implemented by means of two pipeline segments each bent by 90° (collectively 180°).

Also implemented in the preheater segment (F) was a superficial volume ratio of the linear pipeline sections relative to the bent pipeline sections or relative to the deflections, Vₗᵢₙₑₐᵣ/V_{deflection}, according to Table 7. The nominal width of the connecting pipeline elements corresponded to that of the shell of the shell-and-tube apparatuses, and was likewise DN150. This gave a flow rate in these sections according to Table 7.

After successful combination of the two reaction mixtures at 158° C., the resulting mixture was then guided through an adiabatically operated delay segment (G). The delay segment (G) was composed of 13 series-connected linear reactor segments which, in an analogous manner to the preheaters, were arranged parallel to one another and were connected to one another by 180° pipe bends of the same nominal width.

The reactors of the delay segment (G), just like the connecting pipeline elements, were executed with an internal diameter of d, =158 mm and hence result in a flow rate of the reaction mixture according to Table 7. Also implemented in the delay segment (G) was a superficial volume ratio of the linear pipeline sections relative to the bent pipeline sections or relative to the deflections, Vₗᵢₙₑₐᵣ/V_{deflection}, according to Table 7. As a result of the superficial volumes of the preheater segment (F), the delay segment (G) and the connecting pipelines, an average static dwell time according to Table 7 was achieved in the second conversion reactor (F, G).

After the second conversion (F. G), a hot reaction mixture (7a) enriched with methacrylamide (MAA) was obtained, which was then cooled down to 100° C. in a hot water-operated cooler and subsequently fed to a buffer vessel (I) as stream (7b). The average dwell time of the cooled reaction mixture (7b) in the buffer vessel (I) is shown in Table 7.

In the intermediate vessel (I), the gas (9) released in the reaction matrix was removed and combined with the offgases (5a) and (5b) to give the overall offgas (10). In this way, an overall offgas (10) of about 95 m³/h was obtained, which was removed continuously from the process.

A liquid product stream (8) of about 28 700 kg/h was removed continuously from the reaction mixture collected in the intermediate vessel (I), which was analysed by means of HPLC for the methacrylamide (MAA), methacrylic acid (MA) and hydroxyisobutyramide (HIBAm) components. The respective analysis was conducted in triplicate; the respective arithmetic averages are entered in Table 1. Sampling and analysis were effected twice per day, with sampling on 7 successive days in total in steady-state operation of the plant.

The resultant overall yield of components (methacrylamide, methacrylic acid) which are convertible to the target product that was subsequently sent to an esterification for preparation of methyl methacrylate or to a hydrolysis for preparation of methacrylic acid is likewise shown in Table 1. The average yield of 14 representative samples measured in steady-state operation of a process with the conditions specified was 94.1%.

TABLE 1
Results of Comparative Example 1

| | | Concentration in the | | | |
|---|---|---|---|---|---|
| | | product stream [%] | | | Yield [%] |
| Sample | Time | MAA | MA | HIBAm | MAA + MA |
| 1 | Day 1; 10:00 | 35.76 | 1.06 | 0.17 | 94.26 |
| 2 | Day 1; 13:50 | 35.81 | 1.04 | 0.17 | 94.26 |
| 3 | Day 2; 07:40 | 35.75 | 1.03 | 0.15 | 94.09 |
| 4 | Day 2; 11:23 | 35.67 | 1.06 | 0.16 | 93.95 |
| 5 | Day 3; 08:05 | 35.68 | 1.05 | 0.17 | 94.07 |
| 6 | Day 3; 11:05 | 35.71 | 1.06 | 0.16 | 93.95 |
| 7 | Day 4; 09:34 | 35.89 | 1.04 | 0.16 | 94.51 |
| 8 | Day 4; 13:10 | 35.86 | 1.03 | 0.17 | 94.52 |
| 9 | Day 5; 08:40 | 35.79 | 1.05 | 0.16 | 94.28 |
| 10 | Day 5; 11:18 | 35.83 | 1.05 | 0.16 | 94.35 |
| 11 | Day 6; 09:45 | 35.72 | 1.02 | 0.16 | 93.84 |
| 12 | Day 6; 13:00 | 35.86 | 1.04 | 0.16 | 94.51 |
| 13 | Day 7; 08:40 | 35.58 | 1.07 | 0.10 | 93.84 |
| 14 | Day 7; 12:40 | 35.77 | 1.01 | 0.15 | 94.22 |

| | |
|---|---|
| Average yield based on | Ø 94.1 |
| (MAA + MA) | |
| Range of variation of | ±0.59% |
| average yield | |

### Example 2 (Comparative Example): Process for Preparing Methacrylamide in Sulfuric Acid Solution Based on Acetone Cyanohydrin Using 100.6% Oleum

The general procedure for the process with construction of the conversion reactor according to the prior art and using oleum (100.6%) is described hereinafter, with detailed description of the respective process conditions in Tables 2 and 7. Unless stated otherwise, the example was conducted under identical process conditions to those in Example 1.

In Example 2 (comparative example), pipe deflections as shown in FIG. 3 (prior art) were used. The results, which are representative of steady-state operation for 7 days, are shown in Table 2.

A stream of matter (8) that had a concentration of methacrylamide (MAA), methacrylic acid (MA) and hydroxyisobutyramide (HIBAm) according to Table 2 was obtained in the buffer vessel (I).

The average yield of 14 representative samples measured in steady-state operation of the process by means of HPLC, under the conditions specified, was 90.9%.

TABLE 2
Results according to Example 2

| | | Concentration in the | | | |
|---|---|---|---|---|---|
| | | product stream [%] | | | Yield [%] |
| Sample | Time | MAA | MA | HIBAm | MAA + MA |
| 1 | Day 1; 07:22 | 34.75 | 0.14 | 0.02 | 91.60 |
| 2 | Day 1; 10:56 | 34.50 | 0.15 | 0.02 | 90.94 |
| 3 | Day 2; 08:07 | 34.63 | 0.14 | 0.02 | 91.28 |
| 4 | Day 2; 10:05 | 34.54 | 0.13 | 0.06 | 91.05 |
| 5 | Day 3; 07:03 | 34.22 | 0.10 | 0.04 | 90.21 |
| 6 | Day 3; 11:16 | 34.68 | 0.10 | 0.01 | 91.42 |
| 7 | Day 4: 09:49 | 34.67 | 0.13 | 0.10 | 91.39 |
| 8 | Day 4; 12:10 | 34.58 | 0.15 | 0.02 | 91.15 |
| 9 | Day 5; 08:41 | 34.19 | 0.14 | 0.03 | 90.13 |
| 10 | Day 5; 11:53 | 34.04 | 0.14 | 0.03 | 89.73 |
| 11 | Day 6; 08:10 | 34.62 | 0.14 | 0.12 | 91.15 |
| 12 | Day 6; 11:10 | 34.53 | 0.13 | 0.08 | 90.85 |
| 13 | Day 7; 08:21 | 34.21 | 0.09 | 0.04 | 90.60 |
| 14 | Day 7; 10:33 | 34.65 | 0.10 | 0.06 | 91.41 |

| | |
|---|---|
| Average yield based on | Ø 90.9 |
| (MAA + MA) | |
| Range of variation of | ±1.87% |
| average yield | |

### Example 3 (Inventive): Process for Preparing Methacrylamide in Sulfuric Acid Solution Based on Acetone Cyanohydrin with Flow-Optimized Converters

The general procedure for the process with flow-optimized construction of the conversion reactor is described hereinafter, with description of the respective process conditions in Tables 3 and 7. Unless stated otherwise, the process according to the invention was conducted under identical process conditions to those in Example 1.

In Inventive Example 3, pipe deflections as shown in FIG. 4 (inventive) were used. The results, which are representative of steady-state operation for 7 days, are shown in Table 3. The changes in construction according to the invention in the preheater segments (B) and (F) resulted in new flow and reaction conditions in the converters (B, C) and (F, G), which are shown in Table 7. The respective effect on the yield of the process is also shown in Table 3.

A stream of matter (8) that had a concentration of methacrylamide (MAA), methacrylic acid (MA) and hydroxyisobutyramide (HIBAm) according to Table 3 was obtained in the buffer vessel (I).

The average yield of 14 representative samples measured in steady-state operation of the process by means of HPLC, under the conditions specified, was 96.5%.

TABLE 3
Results according to Example 3

| | | Concentration in the | | | |
|---|---|---|---|---|---|
| | | product stream [%] | | | Yield [%] |
| Sample | Time | MAA | MA | HIBAm | MAA + MA |
| 1 | Day 1; 08:45 | 36.33 | 0.95 | 0.23 | 96.60 |
| 2 | Day 1; 10:45 | 36.22 | 0.98 | 0.18 | 96.60 |
| 3 | Day 2; 10:00 | 36.13 | 0.99 | 0.18 | 95.91 |
| 4 | Day 2; 11:30 | 35.57 | 0.97 | 0.14 | 96.69 |
| 5 | Day 3; 10:25 | 36.30 | 1.05 | 0.14 | 96.73 |
| 6 | Day 3; 11:25 | 35.98 | 1.09 | 0.13 | 96.22 |
| 7 | Day 4; 14:35 | 36.42 | 0.90 | 0.23 | 96.83 |
| 8 | Day 4; 16:45 | 36.26 | 0.90 | 0.24 | 96.43 |
| 9 | Day 5; 08:50 | 36.21 | 0.94 | 0.22 | 96.37 |
| 10 | Day 5; 13:00 | 36.39 | 0.93 | 0.20 | 96.71 |
| 11 | Day 6; 09:45 | 36.35 | 0.87 | 0.19 | 96.49 |
| 12 | Day 6; 12:30 | 36.40 | 0.95 | 0.21 | 96.67 |
| 13 | Day 7; 10:00 | 36.38 | 0.87 | 0.24 | 96.64 |
| 14 | Day 7; 12:50 | 35.58 | 0.85 | 0.21 | 96.56 |

| | |
|---|---|
| Average yield based on | Ø 96.5 |
| (MAA + MA) | |
| Range of variation of | ±0.32% |
| average yield | |

### Example 4 (Inventive): Process for Preparing Methacrylamide in Sulfuric Acid Solution Based on Acetone Cyanohydrin with Flow-Optimized Converters and Optimal Sulfuric Acid Concentration

The yield results for the combination of optimal sulfuric acid concentration and flow-optimized construction of the conversion reactors are listed in Table 4 below.

Unless stated otherwise, the process according to the invention was conducted under identical process conditions to those in Example 1. Unlike in Example 1, a sulfuric acid concentration of 99.5% H2SO4 (0.5% water) was used here rather than a sulfuric acid concentration of 99.7%.

In Inventive Example 4, pipe deflections as shown in FIG. 4 (inventive) were used. The results, which are representative of steady-state operation for 7 days, are shown in Table 4. The changes in construction according to the invention in the preheater segments (B) and (F) resulted in nearly identical flow and reaction conditions in the conversion (B. C) and (F, G), which are shown in Table 7.

The average yield of 14 representative samples measured in steady-state operation of the process by means of HPLC, under the conditions specified above, was 97.0%.

TABLE 4
Results according to Example 4

| | | Concentration in the | | | |
|---|---|---|---|---|---|
| | | product stream [%] | | | Yield [%] |
| Sample | Time | MAA | MA | HIBAm | MAA + MA |
| 1 | Day 1; 07:20 | 36.54 | 0.92 | 0.09 | 97.11 |
| 2 | Day 1; 09:15 | 36.36 | 1.00 | 0.21 | 96.79 |
| 3 | Day 2; 09:00 | 36.51 | 0.94 | 0.22 | 97.01 |
| 4 | Day 2; 10:38 | 36.39 | 0.90 | 0.26 | 97.02 |
| 5 | Day 3; 08:40 | 36.47 | 0.93 | 0.25 | 97.04 |
| 6 | Day 3; 10:40 | 36.54 | 0.83 | 0.26 | 97.25 |
| 7 | Day 4; 07:45 | 36.61 | 0.91 | 0.22 | 97.09 |
| 8 | Day 4; 10:40 | 36.57 | 1.03 | 0.17 | 97.09 |
| 9 | Day 5; 08:45 | 36.42 | 1.16 | 0.14 | 97.02 |
| 10 | Day 5; 10:00 | 36.10 | 0.69 | 0.43 | 97.03 |
| 11 | Day 6; 08:00 | 36.23 | 0.75 | 0.33 | 96.65 |
| 12 | Day 6; 10:40 | 36.14 | 0.88 | 0.27 | 96.60 |
| 13 | Day 7; 08:45 | 36.53 | 0.95 | 0.23 | 97.12 |
| 14 | Day 7; 10:45 | 36.43 | 0.98 | 0.18 | 96.95 |

| | |
|---|---|
| Average yield based on | Ø 97.0 |
| (MAA + MA) | |
| Range of variation of | ±0.33% |
| average yield | |

### Example 5 (Inventive): Process for Preparing Methacrylamide in Sulfuric Acid Solution Based on Acetone Cyanohydrin with Flow-Optimized Converters and Optimal Sulfuric Acid Concentration with Elevated Dwell Time and Temperature of the Product Mixture in the Buffer Vessel

In Inventive Example 5, pipe deflections as shown in FIG. 4 (inventive) were used. The results, which are representative of steady-state operation for 7 days, are shown in Table 5. The yield results for the combination of optimal sulfuric acid concentration, flow-optimized converters and elevated dwell time of the product mixture in the buffer vessel (I) are depicted in Table 5 below.

Unless stated otherwise, the process according to the invention was conducted under identical process conditions to those in Example 4. In a departure from the preceding examples, however, the reaction mixture (7b) was cooled to 116° C., and the average dwell time of the mixture (7b) in the buffer vessel (I) was 51 min.

The average yield of 14 representative samples measured in steady-state operation of the process by means of HPLC, under the conditions specified, was 96.4%.

TABLE 5
Results according to Example 5

| | | Concentration in the | | | |
|---|---|---|---|---|---|
| | | product stream [%] | | | Yield [%] |
| Sample | Time | MAA | MA | HIBAm | MAA + MA |
| 1 | Day 1; 07:10 | 35.73 | 0.75 | 0.22 | 96.15 |
| 2 | Day 1; 11:05 | 36.14 | 0.88 | 0.17 | 96.00 |
| 3 | Day 2; 09:00 | 36.33 | 0.95 | 0.17 | 96.40 |
| 4 | Day 2; 10:10 | 36.22 | 0.98 | 0.13 | 96.60 |
| 5 | Day 3; 08:10 | 36.13 | 0.99 | 0.13 | 95.91 |
| 6 | Day 3; 11:20 | 35.57 | 0.97 | 0.12 | 96.49 |
| 7 | Day 4; 07:05 | 36.30 | 1.05 | 0.22 | 96.83 |
| 8 | Day 4; 10:00 | 35.98 | 1.09 | 0.23 | 96.22 |
| 9 | Day 5; 07:40 | 36.42 | 0.90 | 0.21 | 96.83 |
| 10 | Day 5; 10:05 | 36.26 | 0.90 | 0.19 | 96.32 |
| 11 | Day 6; 08:10 | 36.21 | 0.94 | 0.18 | 96.37 |
| 12 | Day 6; 10:55 | 36.10 | 0.93 | 0.20 | 96.39 |
| 13 | Day 7; 08:40 | 36.35 | 0.87 | 0.23 | 96.79 |
| 14 | Day 7; 12:45 | 35.98 | 1.09 | 0.20 | 96.42 |

| | |
|---|---|
| Average yield based on | Ø96.4 |
| (MAA + MA) | |
| Range of variation of | ±0.46% |
| average yield | |

### Example 6 (Inventive): Process for Preparing Methacrylamide in Sulfuric Acid Solution Based on Acetone Cyanohydrin with Flow-Optimized Converters and Optimal Sulfuric Acid Concentration with Reduced Plant Load

In Inventive Example 6, pipe deflections as shown in FIG. 4 (inventive) were used. The results, which are representative of steady-state operation for 7 days, are shown in Table 6. The yield results for the combination of optimal sulfuric acid concentration and flow-optimized converters with reduced plant load are shown in Table 6 below.

The process according to the invention was conducted under the process conditions detailed in Table 7. The plant load mentioned (in %) is based on the respective feed streams (1a+1b) and the sulfuric acid stream (2) which is in a fixed ratio thereto. In a departure from the preceding examples, the plant load in this example was reduced from 100%, corresponding to 11 000 kg/h of acetone cyanohydrin, to 75%, corresponding to 8250 kg/h of acetone cyanohydrin.

The average yield of 14 representative samples measured in steady-state operation of the process by means of HPLC, under the conditions specified, was 96.8%.

In addition, the altered flow conditions in the deflections of the preheater segments (B) and (F) are shown in the CFD simulation in FIG. 3.

TABLE 6
Results according to Example 6

| | | Concentration in the | | | |
|---|---|---|---|---|---|
| | | product stream [%] | | | Yield [%] |
| Sample | Time | MAA | MA | HIBAm | MAA + MA |
| 1 | Day 1; 08:50 | 35.57 | 0.97 | 0.20 | 96.79 |
| 2 | Day 1; 12:25 | 36.30 | 1.05 | 0.19 | 96.83 |
| 3 | Day 2; 09:25 | 35.98 | 1.09 | 0.19 | 96.62 |
| 4 | Day 2; 10:35 | 36.42 | 0.90 | 0.15 | 96.83 |
| 5 | Day 3: 14:10 | 36.26 | 0.90 | 0.16 | 96.62 |
| 6 | Day 3; 16:20 | 36.21 | 0.94 | 0.14 | 96.57 |
| 7 | Day 4; 07:15 | 36.39 | 0.93 | 0.22 | 97.01 |
| 8 | Day 4; 10:50 | 36.35 | 0.87 | 0.23 | 96.79 |
| 9 | Day 5; 07:50 | 36.40 | 0.95 | 0.21 | 97.07 |
| 10 | Day 5; 10:50 | 36.38 | 0.87 | 0.18 | 96.96 |
| 11 | Day 6; 07:10 | 35.58 | 0.85 | 0.18 | 96.79 |
| 12 | Day 6; 10:05 | 36.22 | 0.86 | 0.20 | 96.51 |
| 13 | Day 7; 08:40 | 36.15 | 0.88 | 0.21 | 96.85 |
| 14 | Day 7; 11:55 | 36.29 | 0.88 | 0.20 | 96.79 |

| | |
|---|---|
| Average yield based on | Ø 96.8 |
| (MAA + MA) | |
| Range of variation of | ±0.28% |
| average yield | |

Table 7 summarizes the most important process parameters and results of the examples described.

TABLE 7
Selected process parameters of Examples 1-6

| | | Example | | | | | |
|---|---|---|---|---|---|---|---|
| | | 1′ | 2′ | 3 | 4 | 5 | 6 |
| H₂SO₄ concentration | % | 99.7 | 100.6 | 99.7 | 99.5 | 99.5 | 99.5 |
| Acetone cyanohydrin | % | 99.0 | 99.0 | 99.0 | 99.0 | 99.0 | 99.0 |
| quality | | | | | | | |
| Mass ratio of H₂SO₄/ACH | kg/kg | 1.62 | 1.62 | 1.62 | 1.62 | 1.62 | 1.62 |
| Plant load | % | 100 | 100 | 100 | 100 | 100 | 75 |
| w_{Ø}* in preheater | m/s | 1.22 | 1.23 | 1.22 | 1.22 | 1.22 | 0.91 |
| segment (B) | | | | | | | |
| w_{Ø}* in 90/180° pipe | m/s | 0.12 | 0.13 | 1.05 | 1.05 | 1.05 | 0.78 |
| bends of preheater (B) | | | | | | | |
| w_{Ø}* in delay segment (C) | m/s | 0.23 | 0.24 | 0.23 | 0.23 | 0.23 | 0.17 |
| τ_{Ø}** conversion (B + C) | min | 4.82 | 4.80 | 4.62 | 4.62 | 4.62 | 6.16 |
| Vₗᵢₙₑₐᵣ/V_{deflection} *** in (B) | | 1.1 | 1.1 | 9.8 | 9.8 | 9.8 | 9.8 |
| Vₗᵢₙₑₐᵣ/V_{deflection} *** in (C) | | 28.7 | 28.7 | 28.7 | 28.7 | 28.7 | 28.7 |
| w_{Ø}* in preheater | m/s | 1.48 | 1.49 | 1.48 | 1.48 | 1.48 | 1.13 |
| segment (F) | | | | | | | |
| w_{Ø}* in 90/180° pipe | m/s | 0.14 | 0.15 | 1.28 | 1.28 | 1.28 | 0.97 |
| bends of preheater (F) | | | | | | | |
| w_{Ø}* in delay segment (G) | m/s | 0.28 | 0.29 | 0.28 | 0.28 | 0.28 | 0.21 |
| τ_{Ø}** conversion (F + G) | min | 4.63 | 4.61 | 4.41 | 4.41 | 4.41 | 5.86 |
| Vₗᵢₙₑₐᵣ/V_{deflection} *** in (F) | | 1.5 | 1.5 | 13.7 | 13.7 | 13.7 | 13.7 |
| Vₗᵢₙₑₐᵣ/V_{deflection} *** in (G) | | 31.0 | 31.0 | 31.0 | 31.0 | 31.0 | 31.0 |
| Average dwell time in | min | 12 | 12 | 12 | 12 | 51 | 14 |
| buffer vessel (H) | | | | | | | |
| Temperature in buffer | ° C. | 100 | 100 | 100 | 100 | 120 | 100 |
| vessel (H) | | | | | | | |
| Average yield (MAA + MA) | % | Ø 94.1 | Ø 90.9 | Ø 96.5 | Ø 97.0 | Ø 96.4 | Ø 96.8 |
| based on ACH | | | | | | | |
| Scatter of the yield | % | ±0.59 | ±1.87 | ±0.32 | ±0.33 | ±0.46 | ±0.28 |

′comparative example
*w_{Ø}: average flow rate
**τ_{Ø}: average dwell time including pipelines
*** proportion by volume of linear reactor segments relative to the proportion by volume of bent reactor segments

The results in Tables 1-7 illustrate that the controlled reduction of backmixing in the preheater segments of the respective conversion reactors enables a distinct gain in yield (Example 3) compared to the conventional mode of operation (Example 1 or 2). These optimal yields are subject to much less variation compared to processes that use parameters and apparatuses according to the prior art (cf. Examples 1 and 2).

The effect of the changes in construction of the conversion reactors on the speed distribution in the deflecting pipe elements of the preheater segments were additionally demonstrated and visualized by CFD calculations (FIGS. 3 and 4).

It is also found that the combination of the flow-optimized backmixing-reduced conversion according to the invention in conjunction with an optimal sulfuric acid concentration (99.5%) permits the achievement of a maximum yield (Example 4).

Furthermore, it is also crucial for a maximum yield that the reaction mixture obtained is cooled before being stored intermediately in a buffer vessel (Example 4).

Moreover, the combination of the converter construction according to the invention and the sulfuric acid concentration optimized in accordance with the invention, even in the case of reduced plant load, enables an effective reduction in backmixing-related yield losses, such that high yields can be assured even in partial load operation. More particularly, the process according to the invention enables better control of the dwell time profile of the reaction, even in the case of varying space-time yield within the reactor used. Example 7 (CFD simulation):

The flow conditions in a pipe deflection according to Examples 1 and 2 and Inventive Examples 3-6 were illustrated by CFD (computational fluid dynamics) simulation. For the CFD simulation, a population of 12 000 volume elements was considered, the individual dwell time of which was calculated in the steady flow state. In FIGS. 3 and 4 are each, by way of example, 2 simulation results for different plant loads for the comparative example (prior art) (FIGS. 3 and 5a, and Examples 1 and 2) and for the process according to the invention (FIGS. 4 and 5b, and Examples 3-6).

The qualitative representations of the flow vectors according to the comparative example (right-hand images in FIGS. 3) clearly show regions with significant backmixing (Z_{RV}) in the pipe deflection, which have an adverse effect on the dwell time distribution of the reaction mixture.

This becomes clearer when the cumulative distribution of the pipe deflection of the comparative example (Figure Sa) is considered at different flow rates (load). For example, at 100% load (see Example 1 or 2), 80% of the volume elements considered dwell for about 1.8 seconds in a 180° deflection (see solid line in FIG. 5a). In the case of reduced plant load to 75% (flow-through), there is a distinct rise in this value to 3.2 seconds (see dotted line in Figure Sa). In addition, the average dwell time of the remaining 10% of the reaction mixture is >5 seconds. If the large number of deflections in the preheater segment is taken into account, a much longer dwell time in the reactor is found for at least 10% of the reaction mixture, which ultimately leads to the drops in yield measured.

The results of the flow simulation in a pipe deflection according to the inventive example (FIGS. 4 and Sb) show, by comparison with the comparative example, that it was possible to eliminate the areas with significant backmixing that have an adverse effect on the dwell time distribution of the reaction mixture. This is made clear not only by the qualitative representation of the flow vectors (right-hand images in FIG. 4) but also when the cumulative distribution at different flow rates is considered (FIG. 5b). In the example shown, 80% of the volume elements considered dwell for only 0.55 second at 100% load (cf. Example 3), and for only 1.0 second in a 180° deflection at partial load (75%). Furthermore, the much narrower dwell time distribution of this construction is apparent since the volume elements having a dwell time of >1 second exit from the deflection after no later than 3 seconds, which is not the case, for example, in Comparative Example 1 in FIG. 5a.

1-17. (canceled)

18: A process for preparing methyl methacrylate and/or methacrylic acid, the process comprising:
a. reacting acetone cyanohydrin and sulfuric acid in one or more reactors I in a first reaction stage at a temperature in the range from 70° C. to 130° C., to obtain a first reaction mixture comprising sulfoxyisobutyramide and methacrylamide;
b. converting the first reaction mixture by heating to a temperature in the range from 130 to 200° C., in one or more reactors 11 in a second reaction stage, to obtain a second reaction mixture comprising predominantly the methacrylamide and the sulfuric acid; and
c. reacting the second reaction mixture with water and optionally methanol in one or more reactors III in a third reaction stage, to obtain a third reaction mixture comprising the methacrylic acid and/or the methyl methacrylate:
wherein
(i) the sulfuric acid used in the first reaction stage, which is fed in at one or more points in the one or more reactors I, has a concentration in the range from 98.0% by weight to 100.5% by weight,
(ii) a dwell time of the first reaction mixture in the second reaction stage is in the range from 2 to 15 min,
(iii) the heating in the second reaction stage is performed in the one or more reactors II, wherein at least one reactor II comprises at least one preheater segment comprising one or more linear pipeline elements that are heated by a heating medium spatially separate from the first reaction mixture, wherein the first reaction mixture is heated by 10 to 100° C.,
(iv) the converting in the second reaction stage is performed in the one or more reactors II, wherein at least one reactor II comprises at least one delay segment which is operated under virtually adiabatic conditions,
(v) the at least one preheater segment and/or the at least one delay segment are implemented in a combination of linear pipeline elements and deflections, wherein the linear pipeline elements and the deflections are connected to one another via reducing flanges,
(vi) the second reaction mixture obtained in b, comprising predominantly the methacrylamide and the sulfuric acid, is optionally cooled down to a temperature below 120° C., and/or optionally intermediately buffered in an intermediate vessel, before the second reaction mixture is guided into the third reaction stage, and
wherein a superficial volume of the linear pipeline elements relative to a superficial volume of the deflections in the at least one preheater segment corresponds to a ratio in the range from 2.0 to 75.0.

19: The process according to claim 18, wherein, in the at least one preheater segment, the linear pipeline elements are executed in one or more straight pipelines having an internal diameter in the range from 8 mm to 200 mm, and
wherein pipelines of the linear pipeline elements in the at least one preheater segment have a smaller internal diameter than pipelines of the linear pipeline elements in the at least one delay segment.

20: The process according to claim 18, wherein linear pipeline elements in the at least one preheater segment and/or in the at least one delay segment of the at least one reactor II are executed in parallel bundles of tubes,
wherein the bundles of tubes are connected by at least one deflection, and
wherein the at least one deflection is configured as a pipeline comprising a first section having an internal cross section that runs convergently in flow direction, a second section having an essentially constant internal cross-sectional area, and a third section having an internal cross section that runs divergently in the flow direction.

21: The process according to claim 18, wherein the at least one preheater segment comprises linear pipeline elements and deflections,
wherein the linear pipeline elements of the at least one preheater segment comprise 1 to 50 separate straight pipelines that are secondarily heated with a heating medium and are arranged parallel to one another, and
wherein the deflections that connect the linear pipeline elements to one another comprise at least one pipeline.

22: The process according to claim 18, wherein the deflections are formed from pipeline elements having an inflection of 90° to 180°.

23: The process according to claim 18, wherein an average speed of the first reaction mixture of 0.2 to 3 m/sec is established within the deflections and a variance in a local speed at any point within the deflections from an average conveying speed is not more than 30%.

24: The process according to claim 18, wherein at least one intermediate conversion takes place after performance of the first reaction stage, before a further first reaction stage and the second reaction stage,
wherein at least two reactors II for the at least one intermediate conversion and the second reaction stage are implemented by at least one shell-and-tube heat exchanger as a preheater segment, combined with at least one delay zone as a delay segment.

25: The process according to claim 24, wherein at least two reactors I are used for the first reaction stage and the further first reaction stage, wherein at least one reactor II comprising at least one preheater segment and/or delay segment is arranged between the at least two reactors I, and at least one reactor II comprising at least one preheater segment and/or delay segment is arranged at an exit from the further first reaction stage.

26: The process according to claim 18, wherein at least 60% of static dwell time of at least one second reaction stage is implemented in a dwell segment executed as a pipeline.

27: The process according to claim 18, wherein the first reaction mixture resulting from the first reaction stage, proceeding from the one or more reactors I, is conveyed through the one or more reactors II at a constantly controlled mass flow rate by a discharge pump.

28: The process according to claim 18, wherein the at least one preheater segment in the one or more reactors II is configured as a shell-and-tube heat exchanger, and
wherein the first reaction mixture flows through the shell-and-tube heat exchanger on a tube side, and a free cross-sectional area of tubes in the shell-and-tube heat exchanger ensures an average flow rate of at least 0.2 m/s in the tubes.

29: An apparatus for performance of the process according to claim 18, comprising:
the one or more reactors I, the one or more reactors II, and the one or more reactors III, which are connected to one another by pipeline elements,
wherein at least one reactor II comprises the at least one preheater segment and the at least one delay segment, wherein the at least one preheater segment comprises the one or more linear pipeline elements that are heated by the heating medium spatially separate from the first reaction mixture, and wherein the at least one delay segment is operated under virtually adiabatic conditions,
wherein the at least one preheater segment and/or the at least one delay segment are implemented in a combination of linear pipeline elements and deflections, wherein the linear pipeline elements and the deflections are connected to one another via the reducing flanges, and
wherein the superficial volume of the linear pipeline elements relative to the superficial volume of the deflections in at least one preheater segment corresponds to a ratio in the range from 2.0 to 75.0.

30: The apparatus according to claim 29, wherein, in the at least one preheater segment, the linear pipeline elements are executed in one or more straight pipelines having an internal diameter in the range from 8 mm to 200 mm, and
wherein pipelines of the linear pipeline elements in the at least one preheater segment have a smaller internal diameter than pipelines of the linear pipeline elements in the at least one delay segment.

31: The apparatus according to claim 29, wherein the deflections are formed from pipeline elements having an inflection of 90° to 180°.

32: The apparatus according to claim 29, wherein the linear pipeline elements of the at least one preheater segment and/or of the at least one delay segment are connected by connective deflections, wherein the connective deflections are each an element that causes deflection by 180°, formed from two 900 pipeline deflections and one linear pipeline element.

33: The apparatus according to claim 32, wherein in the connective deflections, the linear pipeline element is disposed between the two 90° pipeline deflections.
